(19) United States
(12) Patent Application Publication
(10) Pub. No.: US 2024/0177987 A1
(43) Pub. Date: May 30, 2024

(54) Method for Analyzing Samples Including a High M/Z Cutoff

(71) Applicant: DH Technologies Development Pte. Ltd., Singapore (SG)

(72) Inventors: Leigh BEDFORD, Concord (CA); Thomas R. COVEY, Concord (CA); James HAGER, Concord (CA); Bradley B. SCHNEIDER, Bradford (CA)

(21) Appl. No.: 18/282,841

(22) PCT Filed: Nov. 18, 2021
(86) PCT No.: PCT/IB2021/060678

§ 371 Date: Sep. 19, 2023

Related U.S. Application Data
(60) Provisional application No. 63/166,158, filed on Mar. 25, 2021.

(51) Int. Cl. H01J 49/42; G01N 33/02; G01N 33/483; H01J 49/24

(52) U.S. Cl. CPC H01J 49/4215; G01N 33/02; G01N 33/4833; H01J 49/24

(57) ABSTRACT

In one aspect, a method of performing mass spectrometric analysis of a sample, e.g. a food-based sample, is disclosed, which comprises ionizing the sample to generate a plurality of ions, introducing the plurality of ions into a mass filter configured to provide a high m/z cutoff greater than a maximum m/z ratio of ions associated with one or more analytes of interest in the sample so as to allow passage of the analyte ions while inhibiting passage of ions having m/z ratios above said high m/z cutoff, and performing a mass analysis of ions passing through said mass filter. In a related aspect, a mass spectrometer is disclosed, which comprises an atmospheric pressure ion source, a first mass filter, a user interface and a controller.

### RELATED APPLICATIONS

The present application claims priority to U.S. Provisional Application No. 63/166,158 filed on Mar. 25, 2021, entitled “Method For Analyzing Samples Including A High M/Z Cutoff,” which is incorporated herein by reference in its entirety.

### BACKGROUND

The present teachings are generally related to mass spectrometric methods and systems for analysis of samples, and more particularly, to such methods and systems that can be employed for analysis of food-based and tissue samples.

Analysis of food-based samples is of increasing importance, in particular in connection with the detection of pesticides and other harmful agents. Such analysis typically requires processing of a sample with reagents to extract the agent(s) (analyte(s)) of interest from a sample under investigation. One common approach for processing of food samples is an extraction method that is known under the acronym QuECheRS (quick, easy, cheap, effective, rugged and safe). This extraction method is typically employed for detection of pesticide residues in food samples. Although it is effective in extracting pesticide residues from a food sample, the QuEChERS approach can result in very complex matrices that can in turn lead to rapid contamination of a mass spectrometer system that is employed to detect the pesticide residue.

For example, when a triple quadrupole mass spectrometer is employed for such analysis, such charge build-up and decrease in performance can be exacerbated in systems that employ low entrance ion energies for the first mass analyzer (e.g., Q1). The contamination of the mass analyzer necessitates periodic cleaning of the system, which can adversely affect the work flow for analysis of the samples and add to the cost of operating the system.

Accordingly, there is a need for enhanced mass spectrometric methods for analysis of samples, and particularly for analysis of food-based samples.

### SUMMARY

In one aspect, a method of performing mass spectrometric analysis of a sample is disclosed, which comprises ionizing a sample to generate a plurality of ions, introducing the plurality of ions into a mass filter positioned upstream of a mass analyzer, wherein said mass filter is configured to provide a high m/z cutoff greater than a maximum m/z ratio of ions associated with one or more analytes of interest in the sample so as to allow passage of the analyte ions while inhibiting passage of ions having m/z ratios above the high m/z cutoff, and performing a mass analysis of ions passing through said mass filter, where the high m/z cutoff is selected so as to reduce contamination of the downstream mass analyzer.

A variety of mass analyzers can be employed. By way of example, and without limitation, the mass analyzer can be a quadrupole mass analyzer (e.g., a triple quadrupole mass analyzer), a time-of-flight (ToF) mass analyzer, an ion trap or combinations thereof.

In some embodiments, at least a portion of ions passing through the mass filter are subjected to fragmentation, e.g., in a collision cell positioned downstream of the mass filter, to generate a plurality of product ions. The product ions can then be subjected to mass analysis, e.g., using a quadrupole and/or a time-of-flight (ToF) mass analyzer, to generate a mass spectrum thereof. In some such embodiments, the step of performing mass analysis can include monitoring one or more multiple reaction monitoring (MRM) transitions of at least one of the analyte ions using a triple quadrupole mass spectrometer.

By way of example, in some embodiments, the high m/z cutoff can be set at about 700, or at about 900 or at about 1000, though other cutoff values can also be employed, e.g., depending on the m/z ratios of contaminating ions.

In some embodiments, the sample under study can be a food-based sample. By way of example, and without limitation, in some such embodiments, such a food-based sample can be, e.g., any beverage such as tea or coffee and crops such as arugula, lettuce, carrots, or any other sample matrix comprising edible materials. In some embodiments, the sample under study can be a tissue sample.

In some embodiments, the bandpass window of the mass filter can be selected to allow the target ions of interest to pass through the mass filter. By way of example, in some embodiments, the bandpass window of the mass filter can be selected to be in a range of about 20 to about 1250, e.g., about 50-900, or any other suitable range for a particular application.

In some embodiments, the sample is processed prior to its ionization. By way of example, the processing of a food-based sample can be performed, e.g., to extract certain analytes of interest, e.g., one or more pesticides. In some embodiments, a processing method known as QuEChERS is employed, though any other suitable processing techniques can also be used. In some embodiments, a tissue sample can be processed to form a homogenate tissue matrix.

A variety of mass filters can be employed in the practice of the present teachings. By way of example, in some embodiments, the mass filter can include a multipole ion guide. In some such embodiments, the multipole ion guide can include a quadrupole rod set extending from a proximal end to a distal end, where the four rods of the quadrupole rod set are arranged relative to one another so as to provide an inlet at the proximal end for receiving ions and an outlet at the distal end through which ions exit the quadrupole rod set. The quadrupole rod set can include a first pair of rods and second pair of rods, wherein each rod is spaced from and extends alongside a central longitudinal axis, and a plurality of auxiliary electrodes that are interposed between the rods of the quadrupole rod set such that the auxiliary electrodes are separated from one another by a rod of the quadrupole rod set and such that each of the auxiliary electrodes is adjacent to a single rod of the first pair of the rods and a single rod of the second pair of rods.

A power supply is coupled to the multipole ion guide operable to provide i) a first RF voltage to the first pair of rods at a first frequency and in a first phase, ii) a second RF voltage to the second pair of rods at a second frequency equal to the first frequency and in a second phase opposite to the first phase, and iii) an auxiliary electrical signal to each of the auxiliary electrodes, wherein the auxiliary electrical signal applied to each of the auxiliary electrodes is substantially identical, and at least one auxiliary RF voltage source operable to apply an RF voltage to the auxiliary electrodes and an auxiliary DC voltage operable to apply a DC voltage to the auxiliary electrodes to prevent transmission of ions having m/z ratios above said cutoff. In some such embodiments, DC voltages can be applied to the auxiliary electrodes such that the DC voltages applied across two pairs of the auxiliary electrodes have opposite polarities and can be symmetrically offset relative to a reference, e.g., a DC offset applied to the ion guide.

In a related aspect, a method of performing mass spectrometric analysis of a sample is disclosed, which comprises ionizing the sample to generate a plurality of ions, introducing the plurality of ions into a mass filter configured to provide a high m/z cutoff greater than a maximum m/z ratio of ions associated with one or more analytes of interest in the sample so as to allow passage of the analyte ions while inhibiting passage of ions having m/z ratios above said high m/z cutoff, and performing a mass analysis of ions passing through the mass filter.

In a related aspect, a mass spectrometer is disclosed, which comprises an atmospheric pressure ion source configured to receive a sample and ionize the sample to generate a plurality of ions, a first mass filter positioned downstream of the ion source for receiving at least a portion of the plurality of ions, a user interface for receiving information from a user regarding one or more m/z ratios or a range of m/z ratios of interest, and a controller in communication with the user interface and the first mass filter for receiving the information from the user interface regarding the m/z ratios or the range of m/z ratios of interest. The controller determines, based on the information received from the user interface, a maximum m/z ratio of interest for mass analysis and adjusts a bandpass window of the first mass filter such that the bandpass window has a high m/z cutoff greater than said maximum m/z ratio.

In some embodiments, the high m/z cutoff is separated from the maximum m/z ratio by a value in the range of about 10 to about 500 m/z.

In some embodiments, the mass filter and a first mass analyzer positioned downstream of the mass filter are disposed in two separate chambers that are maintained at different pressures, where the pressure in the chamber in which the first mass filter is positioned (herein also referred to as the “first chamber”) is greater than the pressure in which the mass analyzer is positioned (herein also referred to as the “second chamber”). For example, the pressure in the first chamber can be at least about 2 times, or at least about 5 times, or at least about 10 times, or at least about 20 times, or at least about 100 times, greater than the pressure in the second chamber. By way of example, in some embodiments, the operating pressure in the first chamber can be in the range of about 2 to about 20 mTorr and the operating pressure in the second chamber can be less than 5×10⁻⁵ Torr.

In some embodiments, a second mass analyzer can be positioned downstream of the first mass analyzer. In some such embodiments of the above mass spectrometer, any of the first mass filter and the first or the second mass analyzer can comprise a plurality of rods that are arranged in a quadrupole configuration. The RF and DC voltages applied to the quadrupole rods can be selected such that the first mass filter exhibits a desired bandpass window and the downstream mass analyzer allows passage of ions with an m/z ratio of interest while inhibiting the passage of the other ions. For example, a controller in communication with at least one RF voltage source and at least one DC voltage source can apply control signals to these voltage sources to set the bandpass window of the first mass filter and the transmission window of the first mass analyzer.

In some embodiments, the frequency of the applied RF voltage can be, for example, in a range of about 100 kHz to about 10 MHz, and the amplitude of the RF voltage (zero-to-peak amplitude) can be, for example, in a range of about 0 volt to about 6000 volts. By way of example, the DC voltage can be employed as a DC resolving voltage to ensure passage of ions having a desired m/z ratio. By way of example, the DC voltage can be in a range of about 5 volts to about 5000 volts.

The controller can be in communication with the first and/or second mass analyzer to allow passage of at least one m/z ratio associated with the ions received from the first mass filter. The controller can also be configured to shift the first and/or second mass analyzer m/z values to allow passage of ions with different m/z ratios received from the first mass filter.

In a related aspect, a method of performing mass spectrometry using a mass spectrometer having a mass filter is disclosed, which comprises performing a mass analysis of a sample to determine a range of m/z ratios associated with one or more compounds of interest in the sample, identifying a maximum m/z ratio associated with said range of m/z ratios, and adjusting a bandpass window of the mass filter such that it exhibits a high m/z cutoff greater than said maximum m/z ratio. Following the adjustment of the bandpass window of the mass filter, a mass analysis of another portion of the sample can be performed using the mass filter with the adjusted bandpass window.

In a related aspect, a mass spectrometer system is disclosed, which comprises an atmospheric pressure ion source configured to receive a sample and ionize the sample to generate a plurality of ions, a first mass filter positioned downstream of the ion source for receiving at least a portion of the plurality of ions, where the first mass filter is configured to allow passage of ions having m/z ratios within a bandpass window. The system can further include a user interface for receiving, for each of a plurality of measurement periods, a target m/z ratio or a target range of m/z ratios for mass analysis during that measurement period.

A controller is in communication with the user interface and the first mass filter, where the controller is configured to determine, for each of the measurement periods, a maximum m/z ratio and to adjust the bandpass window of the first mass filter such that it exhibits a high m/z cutoff greater than the maximum m/z ratio for that measurement period, where the first mass filter exhibits different high m/z cutoffs for at least two different measurement periods.

In a related aspect, a mass spectrometer is disclosed, which comprises an atmospheric pressure ion source configured to receive a sample and ionize the sample to generate a plurality of ions, a first mass filter positioned downstream of the ion source for receiving at least a portion of the plurality of ions, where the first mass filter is configured to allow passage of ions having m/z ratios within a bandpass window. The mass spectrometer can further include a user interface for receiving a maximum m/z ratio of interest from a user, where the controller adjusts a bandpass window of the first mass filter such that the bandpass window has a high m/z cutoff greater than the maximum m/z ratio. In some embodiments, the controller can set the high m/z cutoff to be greater than the maximum m/z ratio of interest by at least about 10 amu.

In some embodiments, the mass spectrometric systems and methods according to the present teachings can be employed to detect one or more of the following pesticides: 1-(2,4 dichlorophenyl)-2-Imidazole (Imazalil metabolit); 1-4(Chlorophenylurea); 1-Naphthalene acetamide (1-NAD); 2,3,5-Trimethacarb; 2,4-Dichlorobenzophenone; 2,4-dimethylaniline (2,4-Xylide); 2,6-Dichlorobenzamide; 2-Hydroxy-Propoxycarbazone; 6-Chlor-3-phénylpyridazin-4-ol; Acephate; Acequinocyl; Acetamiprid; Acetamiprid-N-desmethyl; Acibenzolar-S-Methyl; Acitidione; Alanycarb; Aldicarb; Aldicarb sulfoxide; Aldicarb-sulfone (Aldoxycarb); Aldimorph; Allidichlor; Alloxydim-sodium; Ametoctradin; Ametrynl; Amidithion; Aminocarb; Amisulbrom; Amitraz; Amitrole; Ancymidol; Anilazine; Anilofos; Aramite; Aspon; Asulam; Athidathion; Atrazin, desethyl; Atrazin; desisopropyl; Atrazine; Avermectin; Azaconazole 1; Azadirachtin; Azamethiphos; Azinphos-methyl; Aziprotryn; Azoxystrobin; Barban; Benalaxyl; Bendiocarb; Benfuracarb; Benodanil; Benomyl; Bensulfuron-methyl; Bentazone Methyl; Benthiavalicarb; Benthiavalicarb-Isopropyl; Benzoximate; Benzoylprop-ethyl; Benzthiazuron; BIPC (Chlorbufam); Bispyribac-sodium; Boscalid (Nicobifen); Bromuconazole; BTS 44596; Bufencarb; Bupirimate; Buprofezin; Butamifos; Butocarboxim; Butocarboxim-Sulfoxide; Butoxycarboxim; Buturon; Cadusafos; Carbaryl; Carbendazim Carbetamide; Carbofuran 1; Carbofuran-3-hydroxy; Carboxin; Carpropamid: Chlorantraniliprole; Chlorbromuron; Chloretoxyfos: Chlorfluazuron; Chlorflurenol-Methyl; Chloridazon; Chloroxuron; Chlorpropham: Chlorsulfuron; Chlorthiamid; Chlorthiophos; Chlortoluron; Chromafenozide; Cinidon-ethyl; Cinosulfuron; Clethodim, among other. It should be understood that the application of the present teachings is not limited to the detection and/or analysis of the examples of chemical species and/or compounds provided above. Rather, the present teachings can be employed to detect and/or analyze any chemical species or component present in a sample, e.g., a food-based sample.

In a related aspect, a method of performing mass spectrometric analysis of a sample is disclosed, which comprises ionizing the sample to generate a plurality of ions, introducing said plurality of ions into a mass filter configured to provide a high m/z cutoff greater than a maximum m/z ratio of ions associated with one or more analytes of interest in said sample so as to allow passage of the analyte ions while inhibiting passage of ions having m/z ratios above the high m/z cutoff, and performing a mass analysis of ions passing through said mass filter.

In a related aspect, a mass spectrometer is disclosed, which comprises an atmospheric pressure ion source configured to receive a sample and ionize the sample to generate a plurality of ions, a first mass filter positioned downstream of the ion source for receiving at least a portion of said plurality of ions, a user interface for receiving information from a user regarding one or more m/z ratios or a range of m/z ratios of interest, and a controller in communication with the user interface and the first mass analyzer for receiving the information from the user interface regarding the m/z ratios or the range of m/z ratios of interest, where the controller determines, based on the information received from the user interface, a maximum m/z ratio of interest for mass analysis and adjusts a bandpass window of said first mass analyzer such that said bandpass window has a high m/z cutoff greater than said maximum m/z ratio.

In some embodiments of the above mass spectrometer, the high m/z cutoff is separated from the maximum m/z ratio by a value in a range of about 10 to about 500 amu.

In some embodiments, the first mass filter is disposed in a reduced pressure chamber. In some such cases, the reduced pressure chamber is maintained at a pressure in a range of about 2 mTorr to about 20 mTorr.

In some embodiments, the first mass filter comprises a plurality of rods arranged in a quadrupole configuration. The mass spectrometer can further include a second mass filter that is positioned downstream of the first mass filter for receiving ions transmitted through the first mass filter, said second mass filter having a bandpass window defining a range of m/z ratios that can be transmitted therethrough. The controller can be in communication with the second mass filter to adjust its bandpass window to allow passage of the m/z ratios associated with ions received from the first mass filter. The controller can also be configured to shift the bandpass window of the second mass filter to allow passage of ions having a different m/z ratio received from said first mass filter.

In a related aspect, a method of performing mass spectrometry using a mass spectrometer having a mass filter is disclosed, which comprises performing a mass analysis of a sample to determine a range of m/z ratios associated with one or more compounds of interest in the sample, identifying a maximum m/z ratio associated with the range of m/z ratios, and adjusting a bandpass window of the mass filter such that it exhibits a high m/z cutoff greater than said maximum m/z ratio. The method can further include performing a mass analysis of the sample with said adjusted bandpass window of the mass filter.

In a related aspect, a mass spectrometer system is disclosed, which comprises an atmospheric pressure ion source configured to receive a sample and ionize the sample to generate a plurality of ions, a first mass filter positioned downstream of the ion source for receiving at least a portion of said plurality of ions, where the first mass filter is configured to allow passage of ions having m/z ratios within a bandpass window. A user interface receives, for each of a plurality of measurement periods, a target m/z ratio or a target range of m/z ratios for mass analysis during that measurement period, and a controller in communication with the user interface and the first mass filter is configured to determine, for each of the measurement periods, a maximum m/z ratio and to adjust the bandpass window of the first mass filter such that it exhibits a high m/z cutoff greater than the maximum m/z ratio for that measurement period, where the first mass filter exhibits different high m/z cutoffs for at least two different measurement periods.

In a related aspect, a mass spectrometer is disclosed, which comprises an atmospheric pressure ion source configured to receive a sample and ionize the sample to generate a plurality of ions, a first mass filter positioned downstream of the ion source for receiving at least a portion of said plurality of ions, where the first mass filter is configured to allow passage of ions having m/z ratios within a bandpass window. A user interface receives a maximum m/z ratio of interest from a user and a controller in communication with the user interface and the first mass analyzer receives said maximum m/z ratio of interest and adjusts the bandpass window such that the bandpass window has a high m/z cutoff greater than said maximum m/z ratio.

In a related aspect, a method of performing mass spectrometric analysis of a sample is disclosed, which comprises ionizing a sample to generate a plurality of ions, introducing the plurality of ions into a mass filter positioned upstream of a mass analyzer, where the mass filter includes a plurality of rods to which RF and/or DC voltages can be applied to provide a high m/z cutoff greater than a maximum m/z ratio of ions associated with one or more analytes of interest in the sample so as to allow passage of those analyte ions while at least a portion of the ions having m/z ratios above the high m/z cutoff are deposited on one or more of the rods of the mass filter, thus inhibiting the passage of the high m/z ions to the downstream mass analyzer, and performing a mass analysis of ions passing through said mass filter.

In a related aspect, a method of performing mass spectrometric analysis of a sample is disclosed, which comprises ionizing a sample to generate a plurality of ions, introducing the plurality of ions into a mass filter positioned upstream of a mass analyzer, where the mass filter includes a plurality of rods to which RF and/or DC voltages can be applied to provide a high m/z cutoff greater than a maximum m/z ratio of ions associated with one or more analytes of interest in the sample so as to allow passage of those analyte ions while causing at least a portion of the ions having m/z ratios above the high m/z cutoff to be deposited on one or more of the rods, thus inhibiting the passage of the high m/z ions to the downstream mass analyzer, and performing a mass analysis of ions passing through said mass filter.

In a related aspect, a method of performing mass spectrometric analysis of a sample is disclosed, which comprises introducing a plurality of ions into a mass filter that is positioned upstream of a mass analyzer, where the mass filter is maintained at a pressure higher than the mass analyzer and where the mass filter is configured to provide a high m/z cutoff greater than a maximum m/z ratio of ions associated with one or more analytes of interest in the sample so as to allow passage of the analyte ions while inhibiting passage of ions having m/z ratios above the high m/z cutoff, and delivering the ions passing through the mass filter to the mass analyzer for performing a mass analysis thereof.

In some embodiments, the mass filter includes at least one multipole rod set that is configured for guiding ions through the mass filter and at least one set of rods positioned to capture at least a portion of the ions having m/z ratios above said m/z cutoff.

In a related aspect, a method of performing mass spectrometric analysis of a sample is disclosed, which includes ionizing a sample to generate a plurality of ions, introducing the plurality of ions into an orifice of a mass spectrometer, and mass filtering the ions by capturing ions having m/z ratios above a high m/z cutoff and allowing passage of lower m/z ions to a downstream mass analyzer. The mass filtering of the ions can be accomplished by transmitting the ions through a mass filter having a plurality of electrodes (herein also referred to as sacrificial electrodes or rods) such that the ions having m/z ratios above the m/z cutoff are captured by at least one of the sacrificial electrodes of the mass filter. The method can further include delivering the ions having m/z ratios below the high m/z cutoff to a downstream mass analyzer for mass analysis thereof. In some embodiments, the mass filter is disposed in a chamber that is maintained at a higher pressure than a chamber in which the downstream mass analyzer is positioned. A variety of mass analyzers can be employed. Some examples of such mass analyzers include, without limitation, a time-of-flight (TOF) mass analyzer, a quadrupole mass analyzer, an ion trap and combinations thereof.

Further understanding of various aspects of the present teachings can be obtained by reference to the following detailed description in conjunction with the associated drawings, which are described briefly below.

### BRIEF DESCRIPTION OF THE DRAWINGS

FIG. 1 is a flow chart illustrating various steps of an embodiment of a method according to the present teachings for performing mass spectrometry,

FIG. 2 is another flow chart illustrating various steps of another embodiment of a method according to the present teachings for performing mass spectrometry,

FIGS. 3 and 4 show a mass filter suitable for use in a system according to an embodiment of the present teachings,

FIG. 5A is a partial view of a mass spectrometer in which a mass filter according to an embodiment of the present teachings is incorporated,

FIGS. 5B and 5C show a schematic example of a bandpass window of a mass filter according to the embodiment depicted in FIG. 5A, where the bandpass window covers two or more m/z ratios at a time, for transmission,

FIG. 6 schematically depicts a mass spectrometer in which a system according to the present teachings is incorporated,

FIG. 7 schematically depicts an example of implementation of a controller and/or an analyzer employed in systems and mass spectrometers according to various embodiments of the present teachings,

FIG. 8 shows MRM charging data acquired on a triple quadrupole system after infusion of 30 mL of a food-based sample matrix,

FIGS. 9A and 9B show digital photographs of debris patterns on the Q1 rod sets after infusion of 30 mL of the food-based matrix,

FIG. 10 shows a mass spectrum of a food-based sample as well as a mass spectrum of the same sample that was obtained by filtering all charged species within the Q0 mass filter, except for ions having an m/z ratio in the range of 250-400, which were then introduced into the Q1 analyzer,

FIG. 11 shows the signal intensity for reserpine ions measured initially and after 10 mL increments up to 100 mL of total matrix, using the bandpass window of FIG. 10.

FIGS. 12A and 12B show digital photographs of the Q1 rod set and FIGS. 12C and 12D show one pair of Q0 tee bars after infusion of 100 mL of a food-based matrix in the mass spectrometer, indicating that no substantial build-up of debris was visible on the Q1 rods, despite infusion of 100 mL of the food-based matrix, with the bulk of the debris deposited on the Q0 tee bars,

FIG. 13 shows the mass spectrum of a food-based sample as well as a mass spectrum of the sample when the bandpass of the Q0 mass filter was set to an m/z window spanning the range of 400 to 850 to obtain a mass spectrum of any ions having m/z ratios within this range,

FIG. 14 shows an overlay of a plurality of intensity measurements for reserpine ions taken with 0-100 mL of a food-based matrix sprayed into the mass spectrometer when the bandpass of the Q0 mass filter was set as shown in FIG. 13.

FIGS. 15A and 15B show photographs of the Q1 rods and FIGS. 15C and 15D show photographs of Q0 tee bars, illustrating that the bulk of the debris was deposited onto the Q0 tee bars, greatly reducing the amount of debris on the Q1 rods compared to the control experiment without tee bars filtering,

FIGS. 16A and 16B show digital photographs of one pair of the Q1 rods after infusing 40 mL of the tea/arugula matrix with a bandpass window set to transmit all ions with m/z greater than 720, demonstrating the presence of large deposits on the Q1 rods, similar to the original baseline data taken without using the tee bars,

FIGS. 17A and 17B show mass spectrometry data representing a Q1 scan for a tea/arugula matrix (top pane) and an Asteroid scan for the same matrix (bottom pane),

FIG. 18 shows the mass spectrum of a food-based sample as well as a mass spectrum of the sample when Q0 tee bars were used to set a mass filtering window to eliminate ions having m/z ratios greater than 900,

FIGS. 19A, 19B, 19C, and 19D show the TIC as well as the change in the Q1 peak width over the course of a 5-minute charging experiment after infusing 100 mL of matrix with the bandpass shown in FIG. 18,

FIGS. 20A and 20B show digital photographs of the Q0 tee bars and Q1 rods, respectively.

FIG. 21 mass spectra of a rat liver homogenate matrix for two modes of Q1 scans with and without Q0 filtering,

FIGS. 22A, 22B, and 22C show Q1 charging data after infusion of 40 mL of a rat liver homogenate sample matrix using no Q0 mass filter,

FIGS. 23A, 23B, and 23C show Q1 charging data after infusion of 40 mL of a rat liver homogenate sample matrix with the Q0 mass filter filtering above m/z 400,

FIGS. 24A and 24B show overlays of Q1 data for m/z of 59, taken after each 10 mL infusion of a rat liver homogenate matrix, and

FIGS. 25A, 25B, 25C, and 25D show digital photographs of debris patterns deposited on one set of poles of Q1 rods of a triple quadrupole mass spectrometer after infusion of 40 mL of a rat liver homogenate, where FIGS. 25A and 25B show that substantial debris patterns were deposited when no high mass filtering by tee bars was employed and FIGS. 25C and 25D show no visible debris deposits when the tee bars were set to filter ions with m/z ratios above 400.

### DETAILED DESCRIPTION

The present disclosure is generally directed to methods and systems for performing mass spectrometry in which a mass filter is configured to exhibit a bandpass window characterized by a high m/z cutoff that allows removing unwanted ions, which can otherwise cause contamination of downstream components, e.g., a downstream mass analyzer, and allow passage of target ions of interest to the downstream components. In many embodiments, the methods according to the present teachings are employed to analyze food-based samples, although other samples, such as tissue samples, can also be analyzed. It has been observed that food-based samples can cause degradation in performance of a mass spectrometer, e.g., a triple quadrupole mass spectrometer, within a short period, e.g., after introducing 30-45 mL of a food-based matrix.

The terms “about” and “approximately” are used herein interchangeably to indicate variations that fall within 15%, or 10%, of a numerical value in either direction. Further, as used herein, the term “substantially” refers to a qualitative condition of exhibiting total or near-total extent or degree of a characteristic or property of interest, where a variation, if any, from a complete state is at most 15% or 10%.

The term “food-based sample” as used herein refers to a sample that includes one or more edible ingredients/components, and in many instances, all ingredients and/or components of a food-based sample are edible.

It has been unexpectedly discovered that in many cases ions having large m/z ratios, e.g., m/z ratios greater than 700, are the primary cause of such performance degradation. This finding is unexpected because mass spectra of food-based samples, for example, typically show a multitude of low-mass peaks and very few high mass peaks. Moreover, it has been unexpectedly discovered that the deposition of such ions on the rods of the first mass analyzer is primarily responsible for the degradation of the spectrometer's performance.

Various embodiments discussed below disclose mass spectrometric methods for analysis of samples, e.g., food-based or tissue samples, in which a mass filter having a high m/z cut-off that is greater than the highest m/z of analytes of interest is employed, e.g., upstream of a first mass analyzer, to reduce, and preferably prevent, contamination of the spectrometer's mass analyzers by the matrix components of the processed sample while allowing detection of the analytes of interest. For example, as discussed in more detail below, in some such embodiments, the high m/z cut-off can be set at about m/z 400, or at about m/z 700, or at about m/z 900, or at about m/z 1000, to reduce, and preferably prevent, contamination caused by matrix components. As discussed below, in many embodiments, the use of such high m/z cut-off can result in at least 3-fold extension of the cleaning interval for the mass spectrometer.

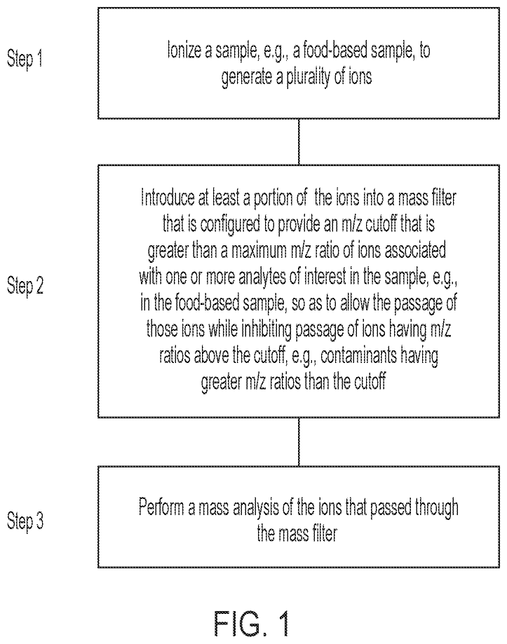

With reference to the flow chart of FIG. 1, in one embodiment of a method according to the present teachings, a sample, e.g., a food-based or a tissue sample, is ionized to generate a plurality of ions (step 1), and at least a portion of the ions is introduced into a mass filter that is configured to provide an m/z cutoff that is greater than a maximum m/z ratio of ions associated with one or more analytes of interest in the sample, e.g., in the food-based sample, so as to allow the passage of those ions while inhibiting passage of ions having m/z ratios above the threshold, e.g., contaminants having greater m/z ratios than the cutoff (step 2). Subsequently, a mass analysis of the ions that passed through the mass filter is performed (step 3).

For example, in some embodiments, the ions passing through the mass filter are introduced into another downstream mass analyzer that is operating at a lower pressure than the pressure at which the upstream mass filter is maintained and is configured for detecting target ions of interest, e.g., ions having a desired m/z ratio. In some embodiments, the m/z cutoff can be set based on the expected m/z ratios of analytes of interest as well as the expected m/z ratios of ions associated with one or more contaminants in the sample.

By way of example, and without any limitation, in some embodiments, the m/z cutoff can be set at about m/z 700 or higher, e.g., at about m/z 720 or at about m/z 1000. For example, in some embodiments in which the food-based sample can include tea and/or arugula, the m/z cutoff can be set at 700 or 1000. For other sample matrices, the m/z cutoff can be selected to be marginally higher than the maximum m/z for the ions of interest, e.g., by about 10 to about 50.

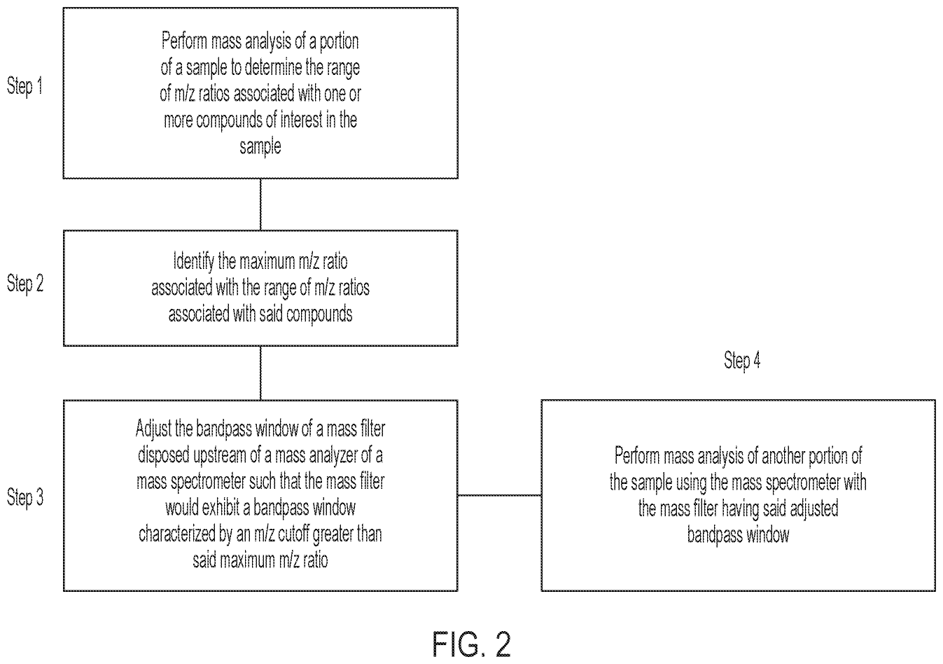

With reference to FIG. 2, in some embodiments, a mass analysis of a sample of interest is performed via mass analysis of a portion of the sample to determine the range of m/z ratios associated with one or more compounds of interest present in the sample (step 1). A maximum m/z ratio of said range of m/z ratios is identified (step 2) and a bandpass window of the mass filter is adjusted such that it would exhibit an m/z cutoff greater than the maximum m/z ratio associated with the analyte ions of interest (step 3). Subsequently, a mass analysis of another portion of the sample with the adjusted bandpass window of the mass filter is performed (step 4). By way of example, in some embodiments, such a mass analysis can be performed using a triple quadrupole mass spectrometer in which the mass filter is positioned upstream of the first mass analyzer of the system.

A variety of mass filters and mass spectrometers can be employed for implementing a method according to the present teachings for mass analysis of samples, and in particular food-based and tissue samples. For example, as discussed in more detail below, the mass filter can include a plurality of rods that are arranged in a multipole configuration and to which RF and/or DC voltages can be applied for radially confining the ions as well as filtering the ions of interest. In some such embodiments, a plurality of auxiliary electrodes can be interposed between the quadrupole rods such that the auxiliary electrodes are separated from one another by one of the quadrupole rods. Further, in some embodiments, the mass filter can be incorporated in a triple quadrupole mass spectrometer or a hybrid quadrupole/time of flight mass spectrometer.

The implementation of methods according to the present teachings for performing mass spectrometry is not limited to the mass filter discussed above. Rather, a variety of mass filters can be employed in the practice of the present teachings. Some examples of suitable mass filters include, without limitation, devices that include tee bars together with a multipole (e.g., quadrupole) arrangement of a plurality of rods, such as the device discussed below, electrical filtering devices, devices for bending the ion path at angles sufficient to eliminate high m/z ratios above a target threshold, among other suitable techniques known in the art.

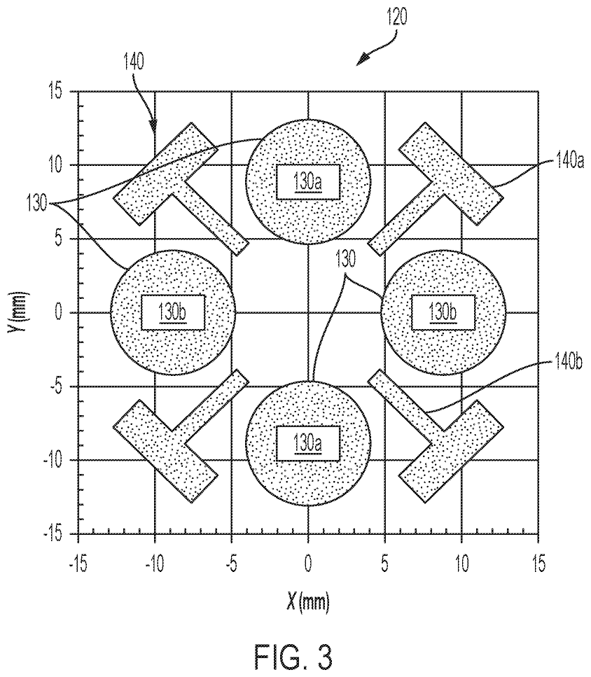

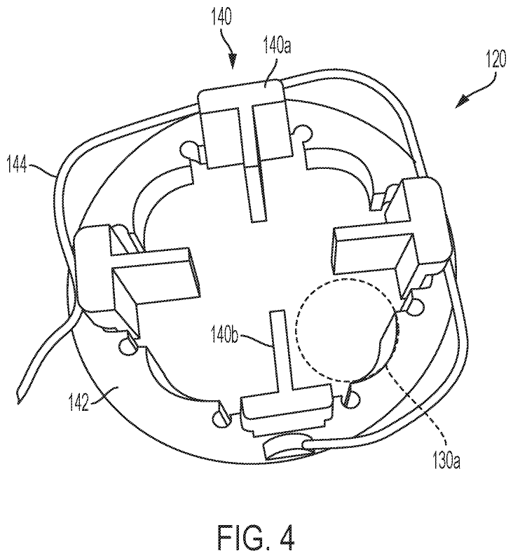

By way of example, in some embodiments, a mass filter described in U.S. Pat. No. 10,741,378 (the “'378 Patent”) entitled “RF/DC Filter to Enhance Mass Spectrometer Robustness,” which is herein incorporated by reference in its entirety, can be employed. Briefly, FIGS. 2 and 3 of the '378 Patent, which are reproduced herein as FIGS. 3 and 4, describe an ion guide 120 that includes a set of four rods 130a,b that extend from a proximal, inlet end disposed adjacent the inlet orifice to a distal, outlet end that is disposed adjacent the exit aperture.

The rods 130a,b are arranged according to a quadrupole configuration to form a quadrupole rod set 130 that surrounds a space through which ions can travel from the inlet end to the outlet end. Similar to the previous embodiment, each of the rods 130 can be electrically coupled to an RF power supply (not shown in FIGS. 3 and 4) such that the rods on opposed sides of the central axis together form a rod pair to which a substantially identical RF signal is applied, and the phase of the RF signal applied to one rod set is opposite to the respective phase of the RF signal applied to the other rod set. A DC offset voltage can also be applied to the rods of the quadrupole rod set.

With continued reference to FIGS. 3 and 4, the ion guide 120 additionally includes a plurality of auxiliary electrodes 140 that are interspersed between the rods of the quadrupole rods of the quadrupole rod set 130. Each of the auxiliary electrodes 140 can be coupled to an RF and/or DC power supply for providing an auxiliary electrical signal thereto so as to control transmission of ions through the ion guide 120. For example, in some embodiments, a DC voltage equal to the DC offset voltage applied to the rods of the quadrupole rod set can be applied to the auxiliary electrodes. Further details regarding various aspects of such mass filters employing auxiliary electrodes can be found in Published International Application No. WO/2020/039371 entitled “RF/DC cutoff to reduce contamination and enhance robustness of mass spectrometry systems,” which is herein incorporated by reference in its entirety.

In some embodiments, a mass filter can be employed that has a bandpass window in the mass-to-charge (m/z) ratio domain that covers multiple m/z ratios, which are transferred to the downstream mass filter. In many embodiments, the bandpass window of the downstream mass filter is configured to allow passage of one m/z ratio at a time. By configuring the bandpass window of the upstream mass filter to allow ions across multiple m/z ratios to be transmitted, a more rapid analysis of a plurality of ions with different m/z ratios can be achieved, as discussed in more detail below.

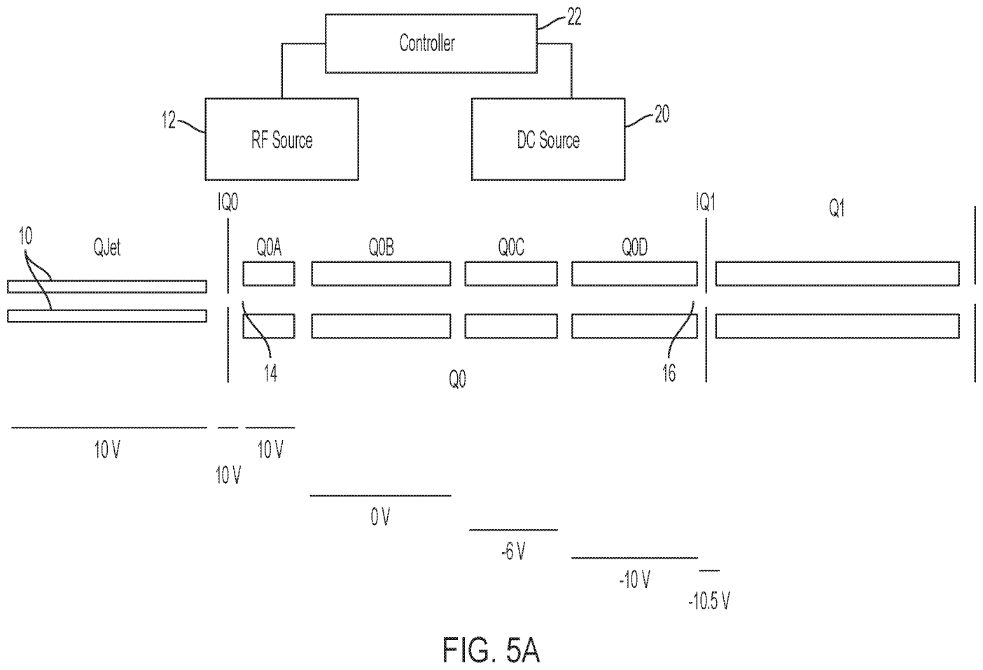

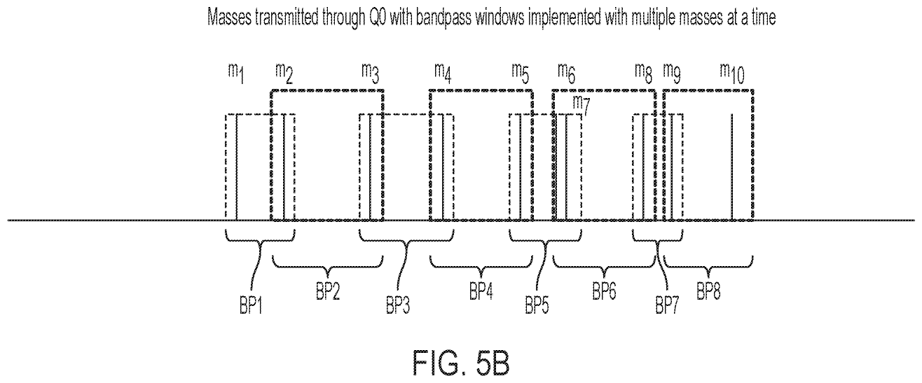

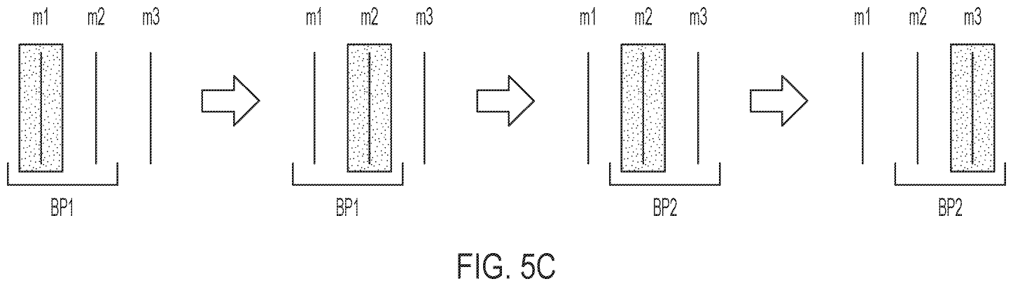

For example, with reference to FIG. 5A, a mass filter Q0 includes four sets of rods Q0A, Q0B, Q0C, and Q0D, which are positioned in series relative to one another in a quadrupole configuration to provide a passageway through which ions received via an inlet 14 of the mass filter can propagate to its outlet 16 through which the ions exit the mass filter Q0. In this embodiment, the Q0 mass filter receives ions from an upstream ion guide QJet that includes four rods 10 (only two of which are visible) that are arranged in a quadrupole configuration.

The RF voltage source 12 (or a separate RF voltage source) and a DC voltage source 20 apply RF and DC voltages to the rods of the mass filter Q0 so as to provide radial focusing of the ions as well as establish a bandpass window (i.e., a transmission window) for passage of ions through the mass filter Q0.

In other words, ions having m/z ratios that fall within the bandpass window of the mass filter Q0 can pass through the mass filter Q0 while transmission of ions having m/z ratios that fall outside the bandpass window is substantially reduced, and preferably inhibited. In this embodiment, RF voltages (signals) are applied to the first, second, and fourth sets of rods Q0A, Q0B, and Q0D, while an RF voltage as well as a resolving DC voltage (for setting the bandpass window of the mass filter Q0) are applied to the third set of rods Q0C, as discussed in more detail below.

In some such embodiments, the bandpass window of the mass filter Q0 (e.g., a quadrupole mass filter) can be configured to include the mass of a precursor ion of interest plus the mass of the next precursor to be monitored. This is shown schematically in FIGS. 5B and 5C. In this example, initially, the mass filter Q0 is configured to have a bandpass window (BP1) that allows transmission of ions with m/z ratios of m₁ and m₂, and the mass analyzer Q1 is configured to allow transmission of ions with m/z ratio of m₁. When the transmission window of the mass analyzer Q1 is shifted to the next mass of interest, i.e., ions with m/z ratio of m₂, the bandpass window of the mass filter Q0 is adjusted to allow transmission of ions with m/z ratio of m₂ as well as ions with m/z ratio of m₃ (this adjusted bandpass window is designated herein as BP2).

In many embodiments, such adjustment of the bandpass window of the mass filter Q0 can be accomplished by first increasing the RF voltage applied to the mass filter Q0 followed by adjusting the resolving DC component. This can result in an initial increase in the bandpass window of the mass filter Q0 to cover both m₂ and m₃, without disrupting the flow of m₂ ions into the downstream mass analyzer Q1. Following the change in the RF voltage of the mass filter Q0, the DC resolving voltage applied to the mass filter Q0 is adjusted so as to tailor its bandpass window to a desired width.

In many such embodiments, the mass analyzer Q1 is operated to select ions with m/z ratio of m₃ while the DC resolving voltage applied to the mass filter Q0 is being adjusted. Subsequently, the bandpass window of the mass filter Q0 can be adjusted to cover m/z ratios of m₃ and m₄ (i.e., bandpass window designated as BP3). In this example, this is followed by adjusting the bandpass window of the mass filter Q0 to cover m₄ and m₅ (See, bandpass window designated as BP4).

In this embodiment, the RF and the DC voltage sources 12 and 20 are operated under the control of a controller 22 to control the application of RF and DC voltages to the mass filter Q0 as well as a mass analyzer Q1, which is positioned downstream of the mass filter Q0, so as to set and adjust the bandpass window of the mass filter Q0 and the transmission window of the mass analyzer Q1. More specifically, the controller 22 can be programmed to control the RF and the DC voltage sources such that the RF and DC voltages that are applied to the rods of the mass filter Q0 and the mass analyzer Q1 provide a desired bandpass window for the mass filter Q0 and also allow transmission of ions having a desired m/z ratio through the mass analyzer Q1. Further, the controller 22 can update the bandpass window of the mass filter Q0 to the next bandpass window and also adjust the RF and/or DC voltages applied to the mass analyzer Q1 to switch the transmission of ions through the mass analyzer Q1 from one m/z ratio to another.

For example, at the beginning of a measurement cycle, the controller 22 can set the bandpass window of the mass filter Q0 and the transmission window of the mass analyzer Q1 such that the bandpass window of the mass filter Q0 would cover multiple m/z ratios of interest and the transmission window of the mass analyzer Q1 would cover one of those m/z ratios. After a preset period of time (e.g., the time required for the mass analyzer Q1 to process the ions having the m/z ratios of interest), the controller 22 switches the transmission window of the mass analyzer Q1 to the next m/z ratio of interest, which is already within the bandpass window of the mass filter Q0, and also shifts the bandpass window of the mass filter Q0 to cover, in addition to the m/z ratio that is being processed by the mass analyzer Q1, a new m/z ratio of interest, e.g., based on a predefined list of m/z ratios of interest that was previously provided to the controller.

In some embodiments, the controller 22 can be configured to shift the transmission bandwidth of the mass filter Q0 and the transmission window of the mass analyzer Q1 substantially concurrently. In other embodiments, the controller 22 can be configured to shift the transmission bandwidth of the mass filter Q0 before shifting the transmission window of the mass analyzer Q1 to the next m/z ratio of interest. For example, referring again to FIG. 5B, while the mass analyzer Q1 is monitoring ions with an m/z of m₂, the controller 22 can shift the transmission bandwidth of the mass filter Q0 to cover ions with m/z ratios of m₂ and m₃ (i.e., from BP1 to BP2). The controller 22 can then shift the transmission window of the mass analyzer Q1 to cover the m/z ratio of m₃ while ions with an m/z ratio of m₃ are equilibrating, e.g. via collisional cooling, in the mass filter Q0.

In the above examples, it has been described that the controller 22 shifts the transmission bandwidth of the mass filter Q0 and the transmission window of the mass analyzer Q1 in the direction of increasing m/z ratios. However, the present teachings are not limited thereto, and in some embodiments, the controller 22 can be configured to shift the transmission bandpass of the mass filter Q0 and the transmission window of the mass analyzer Q1 in the direction of decreasing m/z ratios.

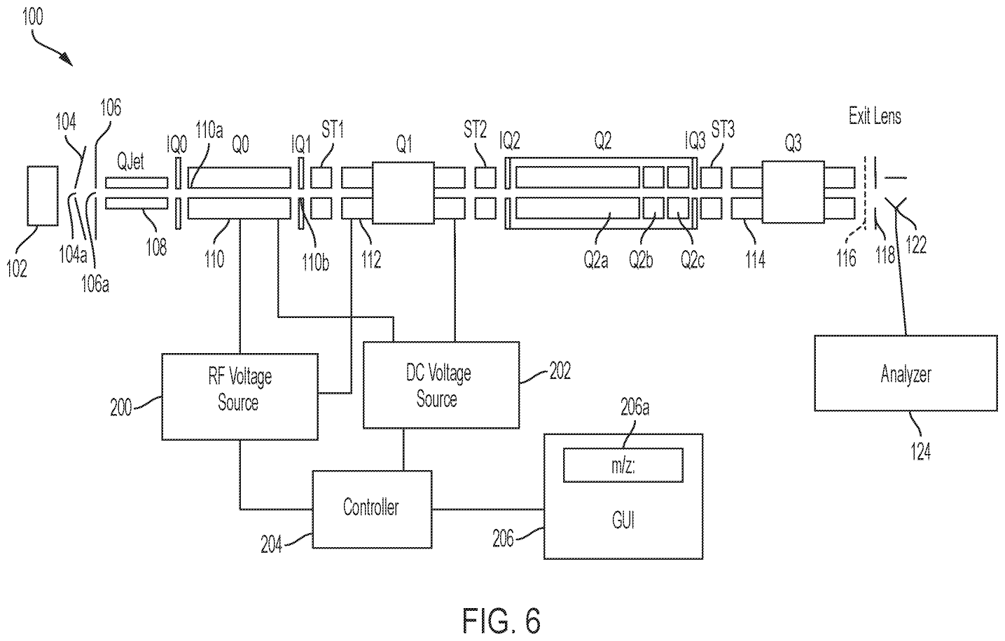

The present teachings can be incorporated in a variety of different mass spectrometers. By way of example, FIG. 6 schematically depicts a mass spectrometer 100, which includes an ion source 102 for generating a plurality of ions. A variety of ion sources can be employed in the practice of the present teachings. Some examples of suitable ion sources can include, without limitation, an electrospray ionization device, a nebulizer assisted electrospray device, a chemical ionization device, a nebulizer assisted atomization device, a chemical ionization device, a matrix-assisted laser desorption/ionization (MALDI) ion source, a photoionization device, a laser ionization device, a thermospray ionization device, an inductively coupled plasma (ICP) ion source, a sonic spray ionization device, a glow discharge ion source, an atmospheric pressure chemical ionization source (APCI), and an electron impact ion source, among others.

The generated ions pass through an aperture 104a of a curtain plate 104 and an orifice 106a of an orifice plate 106, which is positioned downstream of the curtain plate 104 and is separated from the curtain plate 104 such that a gas curtain chamber is formed between the orifice plate 106 and the curtain plate 104. A curtain gas supply (not shown) can provide a curtain gas flow (e.g., of nitrogen) between the curtain plate 104 and the orifice plate 106 to help keep the downstream sections of the mass spectrometer clean by declustering and evacuating large neutral particles. The curtain chamber can be maintained at an elevated pressure (e.g., a pressure greater than the atmospheric pressure) while the downstream sections of the mass spectrometer can be maintained at one or more selected pressures via evacuation through one or more vacuum pumps (not shown).

In this embodiment, the ions passing through the orifices 104a and 106a of the curtain plate 104 and the orifice plate 106 are received by an ion optic QJet, which comprises four rods 108 (two of which are visible in this figure) that are arranged in a quadrupole configuration to form an ion beam for transmission to downstream components of the mass spectrometer 100. In use, the ion optic QJet can be employed to capture and focus the ions received through the opening of the orifice plate 106 using a combination of gas dynamics and radio frequency fields.

The ion beam exits the ion optic QJet and is focused via a lens IQ0 into a subsequent differentially pumped vacuum stage with an additional ion guide (Q0) that can include a mass filter. In some embodiments, the pressure of the mass filter Q0 can be maintained, for example, in a range of about 2 mTorr to about 20 mTorr.

The mass filter Q0 includes four rods 110 (two of which are visible in this figure), which are arranged according to a quadrupole configuration to provide a passageway therebetween that extends from an inlet 110a through which ions can enter the passageway to an outlet 110b through which ions can exit the passageway. As noted above, in this embodiment, the mass filter Q0 receives, via the ion lens IQ0, the ions exiting the ion optic Qjet.

An RF voltage source 200 applies RF voltages to the rods 110 of the mass filter Q0 so as to generate an electromagnetic field within the passageway that can provide radial confinement of the ions as the ions pass through the passageway. In this embodiment, the RF voltage applied to one pair of the rods has the same amplitude and an opposite phase relative to the RF voltage applied the other pair of the rods.

Further, a DC voltage source 202 can apply a resolving DC voltage to at least one of the rods 110 of the mass filter Q0 for setting a bandpass window of the mass filter. In particular, in many embodiments, the resolving DC voltage can be selected to ensure that the bandpass window of the mass filter would exhibit a high m/z cutoff greater than a desired threshold, e.g., 700 or 1000 in some embodiments. Alternatively, the DC voltage source can be used to apply DC potentials to additional tee bar electrodes between the Q0 quadrupole rods. The DC potential can be used to establish a high m/z cut-off in the mass filter. In some embodiments, the high m/z cut-off is fixed at a value higher than the highest m/z of interest, so there is no need to change the mass filter settings during subsequent mass analysis steps.

A controller 204 in communication with the RF and DC voltage sources can control these voltage sources to apply the requisite RF and DC voltages to the rods or auxiliary electrodes. For example, in some embodiments, information regarding the desired characteristics of the mass filter, e.g., the high m/z cutoff value, can be provided to the controller, which can then utilize this information to compute the RF and DC voltages required to achieve the desired characteristics of the mass filter. In many embodiments, the RF frequency can be in a range of about 100 kHz to about 10 MHz and the RF amplitude (zero-to-peak) can be, for example, in a range of about 0 to about 6000 volts.

With continued reference to FIG. 6, in some embodiments, a graphical user interface (GUI) 206 is operably coupled to the controller 204, where the GUI allows a user to input a target m/z ratio of interest or a target range of m/z ratios of interest. By way of example, the GUI can present one or more graphical elements, such as an input window 206a, which allows a user to input information regarding a specific m/z of interest or a range of m/z ratios of interest.

The graphical user interface can convey this information to the controller. In some such embodiments, the controller is programmed to identify the maximum m/z ratio of interest and apply one or more control signals to the RF and/or DC voltage sources so as to set the bandpass window of the mass filter such that the bandpass window has a high m/z cutoff greater than the maximum m/z ratio identified by the controller. By way of example, in some embodiments, the controller can set the high m/z cutoff at a value that is at least 10%, or at least 20%, or at least 30%, or at least 40%, or at least 50%, greater than the maximum target m/z ratio of interest.

The GUI 206 can be implemented in a variety of different ways. Further, in some embodiments, the GUI 206 can present various ways of entering input data based on particular ways in which a mass spectrometer is being operated. For example, in one operational mode, an operator can operate a triple quadrupole mass spectrometer with a first quadrupole mass analyzer set at a specific m/z ratio, such as for example in multiple reaction monitoring mode (MRM), where a second quadrupole functioning as a mass analyzer is also set at a specific m/z ratio corresponding to a fragment ion of interest generated in a collision cell via fragmentation of a plurality of precursor ions. In such an operational mode, the user can enter a series of Q1/Q3 mass pairs into the GUI 206 and the controller 204 can determine the highest m/z ratio of interest and can configure the mass filter to exhibit an m/z cut-off at a value higher than the identified maximum m/z ratio of interest. The m/z cut-off value may be for instance 75 m/z higher, or 150 m/z higher, or any value higher than the maximum m/z ratio of interest. With this approach, a fixed high m/z cut-off is used for all transitions.

In another operational mode, similar to the previous one, a user can enter a series of Q1/Q3 mass pairs, but rather than the controller defining the high m/z cut-off of a mass filter positioned upstream of the mass analyzers, the user can specify the value for the high m/z cut-off. For example, for a food-based analysis, the high m/z cut-off may be a value around m/z 900, though the high m/z cut-off can be different for other samples. The user may also specify a value that is lower than some of the Q1 values. For these MRM transitions, the software and/or firmware will recognize that the specified cut-off would eliminate the signal for some MRM transitions, and it will turn off the tee bars potentials specifically for only those MRM transitions with higher Q1 m/z. With this approach, a fixed high m/z cut-off is used for all transitions with m/z lower than the cut-off.

In yet another operational mode, a user can enter a series of Q1/Q3 mass pairs into the GUI 206 and the software and/or firmware will consider the Q1 values for each pair and will set the conditions for an upstream mass filter to provide a high m/z cut-off relative to the Q1 m/z value, where the offset of the cut-off relative to the Q1 m/z value can be, for example, 75 m/z higher, 150 m/z higher, etc. The user may also specify what offset is desired for each pair.

As discussed above, it has been discovered that such a high m/z cutoff can inhibit certain contaminant ions from reaching the downstream components of the mass spectrometer, such as, a downstream mass analyzer Q1. More specifically, as a result of the high m/z cutoff of the Q0 mass filter, the ions with m/z ratios above the cutoff will acquire unstable trajectories as they pass through the Q0 mass filter and hence are deposited on one or more rods of the Q0 mass filter. Consequently, such ions will not reach the downstream Q1 mass analyzer. However, if Q0 tee bars as described above in connection with FIGS. 3 and 4 are used, ions with m/z values above the cutoff will deposit on the tee bars rather than on the Q0 rods.

It has been unexpectedly discovered that the deposition of the contaminant ions on either the rods of the Q0 mass filter or the tee bars will not adversely affect passage of the target ions through the Q0 mass filter. Without being limited to any particular theory, this phenomenon can be explained via the recognition that the Q0 mass filter is operated typically in the millitorr pressure region in which the collision frequency of the ions with the background neutral species is relatively high, thereby reducing the radial oscillation of the ions that are transmitted through the filter. In other words, the Q0 mass filter operates in the collisional focusing regime. Such radial confinement of the transmitted ions renders those ions less susceptible to contamination of the Q0 rods or the tee bars.

The mass filter Q0 delivers the ions, via an ion lens IQ1, and a stubby lens ST1, which functions as a Brubaker lens, to the downstream mass analyzer Q1. In this embodiment, the mass analyzer Q1 includes four rods 112 (two of which are visible in this figure), which are arranged according to a quadrupole configuration and to which RF and DC voltages can be applied to select an m/z ratio of interest for mass analysis.

The RF voltage source 200 can apply RF voltages to the rods of the Q1 mass filter to cause radial confinement of the ions passing through the mass filter and the DC voltage source 202 can apply a resolving DC voltage to the rods of the Q1 mass analyzer to set the bandpass of the mass analyzer so as to allow the passage of ions having a target m/z or m/z within a target window while inhibiting the passage of ions having other m/z ratios.

The controller 204 can control the RF and DC voltages generated by the RF and DC voltage sources. In particular, the controller can sweep the amplitude of the DC resolving voltage so as to change the bandpass of the mass filter to allow ions with different m/z ratios to pass through the mass filter to be subjected to mass analysis by downstream components of a mass spectrometer in which the mass filter Q0 and the mass analyzer Q1 are incorporated.

The Q1 mass analyzer operates at a lower pressure than the Q0 mass filter. For example, the pressure of the Q0 mass filter can be at least 10 times, or at least 20 times, or at least 30 times, or at least 40 times, or at least 50 times, or at least 60 times, or at least 70 times, or at least 80 times, or at least 90 times, or at least 100 times greater than the pressure of the Q1 mass analyzer. For example, the Q1 mass analyzer can operate at a pressure of less than about 5e⁻⁵ Torr, or about 120 times lower than a typical operating pressure of the Q0 mass filter. Such a reduced pressure results in significantly less collisions between the ions and the background neutral species, thereby leading to a larger radial extent of oscillation for ions passing through the Q1 mass analyzer. This can in turn render the ions more susceptible to charged debris, if any, deposited on the rods of the Q1 mass analyzer. For example, quadrupole mass analyzers typically operate at high Mathieu a-, and q-parameters and narrow transmission windows, e.g., around 1 amu.

Such operating parameters of the quadrupole mass analyzer can require precise setting of both RF and DC voltages. Narrow-band passage of ions through a quadrupole mass analyzer can result in relatively high radial amplitudes of the transmitted ions and sharp distinctions between stable and unstable ions. As a result, any charged material deposited on the rods of the Q1 mass analyzer will “blurr” the boundary between stable and unstable ions by producing a time variation in one or both of the RF and DC fields experienced by the ions passing through the mass analyzer, which can in turn degrade the performance of the mass analyzer.

More specifically, in this embodiment, the quadrupole rod set of the mass analyzer Q1 can be operated as a transmission RF/DC quadrupole mass analyzer for selecting ions having m/z ratios of interest. By way of example, the quadrupole rod set of the mass analyzer Q1 can be provided with RF/DC voltages suitable for operation in a mass-resolving mode. For example, parameters of applied RF and DC voltages can be selected so that the mass analyzer Q1 establishes a transmission window of a chosen m/z ratio, such that those ions can traverse the mass analyzer Q1 largely unperturbed. Ions having m/z ratios falling outside the window, however, do not attain stable trajectories within the quadrupole and can be prevented from traversing the quadrupole rod set of the mass analyzer Q1. It should be appreciated that this mode of operation is but one possible mode of operation for the mass analyzer Q1.

It will be understood by those having ordinary skill in the relevant arts that the energy of ions that are introduced into quadrupole mass analyzers is typically low in order to ensure that the ions can be exposed to sufficient cycles in the quadrupole field as they traverse the quadrupole mass analyzer. By way of example, typical ion energies can be on the order of about 0.5 to about 3 eV and such low-energy ions can be adversely affected by debris accumulation and charging at the quadrupole inlet.

In this embodiment, the ions selected by the mass analyzer Q1 are focused via a stubby lens ST2 and an ion lens IQ2 into a collision cell Q2. In this embodiment, the collision cell Q2 includes a pressurized compartment that can be maintained, e.g., at a pressure in a range of about 1 mTorr to about 20 mTorr, though other pressures can also be used for this or other purposes. A suitable collision gas (e.g., nitrogen, argon, helium, etc.) can be provided by way of a gas inlet (not shown) to cause fragmentation of at least a portion of the ions received by the collision cell Q2.

In this embodiment, the collision cell Q2 includes four rods Q2a that are arranged in a quadrupole configuration and to which RF voltages can be applied to provide radial confinement of the ions received by the collision cell Q2. Further, in this embodiment, a pair of stubby lenses Q2b and Q2c focus the product ions generated via fragmentation of at least a portion of the precursor ions into the orifice of an exit ion lens IQ3 through which the product ions exit the collision cell. It will be apparent to those having ordinary skill in the relevant arts that other embodiments may also be used, for instance, without stubby lenses Q2b and Q2c. The collision cell, Q2, can also include higher order multipoles or ring guides.

The product ions generated by the collision cell Q2 are received by a downstream quadrupole mass analyzer Q3 via an ion lens IQ3 and a stubby lens ST3, which function to focus the product ions into the quadrupole mass analyzer Q3. Although in this embodiment the downstream analyzer is a quadrupole mass analyzer, in other embodiments it can be another type of mass analyzer, e.g., a time-of-flight (ToF) mass analyzer or an ion trap.

The quadrupole mass analyzer Q3 includes four rods 114 that are arranged relative to one another in a quadrupole configuration and to which RF and/or DC voltages can be applied in a manner known in the art to provide mass analysis of the product ions. The ions passing through the mass analyzer Q3 are received and detected, after passage through ion lenses 116 and 118, by a downstream detector 122, which generates ion detection signals in response to the incident ions. An analyzer 124 in communication with the detector 122 receives the ion detection signals and processes the ion detection signals to generate a mass spectrum of the product ions, thereby allowing monitoring of MRM transitions by fixing Q1 on a precursor m/z of interest, fragmenting the precursor ion in Q2, and fixing Q3 on a daughter m/z of interest.

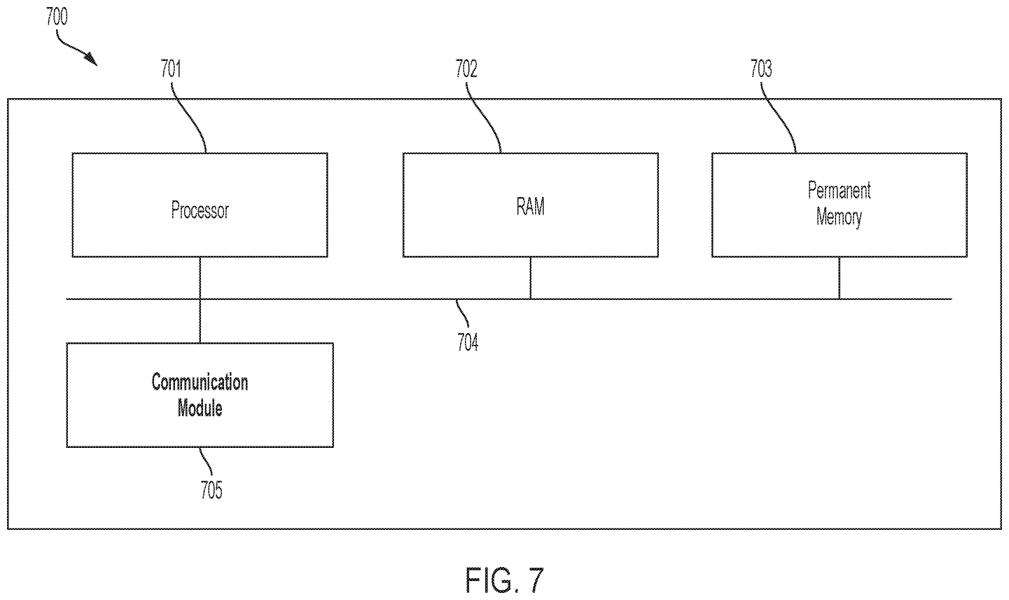

As is known in the art, the analyzer 124 and the controller 204 can be implemented in hardware/firmware and/or software using techniques known in the art as informed by the present teachings. For example, the analyzer 124 can include a processor, one or more random access memory (RAM) modules, one or more permanent memory modules and at least one communication bus for allowing communication among these and other components. By way of example, FIG. 7 schematically depicts an example of implementation 700 of any of the analyzer and the controller, which includes a processor 701, a random access memory (RAM) 702, a permanent memory 703 (e.g., ROM), a communication module 705, and a communications bus 704 that connects the processor to these components. In some embodiments, the instructions for controlling the RF and/or DC voltage sources and/or for analyzing the detection signals by a detector of a mass spectrometer according to the present teachings can be stored in the permanent memory and transferred to the RAM module by the processor during runtime to be executed.

The following Examples are provided for further illustration of various aspects of the present teachings, and are not provided necessarily to show optimal ways of practicing the present teachings and/or optimal results that may be obtained.

### EXAMPLES

A series of experiments were conducted to determine the cause of degradation of the performance of a mass spectrometer when used to analyze food-based samples. As discussed above and further illustrated below, it was unexpectedly discovered that heavy ions, e.g., ions with an m/z ratio greater than about 700, rather than lighter ions, are the primary cause of such degradation in performance. Further, it was discovered that the deposition of charged ions in the first mass analyzer positioned after one or more mass filters can lead to the charging of the rods of the mass analyzer, which can in turn degrade the performance of the mass analyzer. As discussed above, placing a mass filter having a high m/z cutoff upstream of the first mass analyzer can advantageously inhibit passage of such contaminating ions to the first mass analyzer.

The sample matrix included extracts of black tea and arugula. A stock solution of tea was prepared by adding 10 mL of LC/MS grade deionized water to 4 g of tea. The sample was homogenized by shaking for 30 s and then 10 mL of LC/MS grade acetonitrile was added. The sample was vortexed for 10 minutes and a salt mixture comprising 4 g of magnesium sulfate, 1 g of sodium chloride, 1 g of trisodium citrate dehydrate, and 0.5 g of disodium hydrogen citrate sesquihydrate was added. The sample was vortexed for 10 minutes and then centrifuged for 5 minutes at 3500 rpm. The acetonitrile layer was withdrawn and pooled for parallel samples prepared in the same manner.

A similar procedure was used to prepare the arugula stock solution, using 10 g of starting material. A mixture solution was prepared by combining 56 mL of tea extract with 56 mL of arugula extract and adding 28 mL of water and 0.14 mL of formic acid. The mixture solution was diluted 50× with 1:1 acetonitrile:water with 0.1% formic acid, and filtered through a Whatman glass microfiber filter (grade 696).

### Example 1

A triple quadrupole mass spectrometer, similar to that depicted in FIG. 6 above, was used to conduct a series of experiments with extracts of tea and arugula to characterize the signal degradation due to contamination and the m/z range of charged species that produce quadrupole analyzer deposits sufficient to reduce performance.

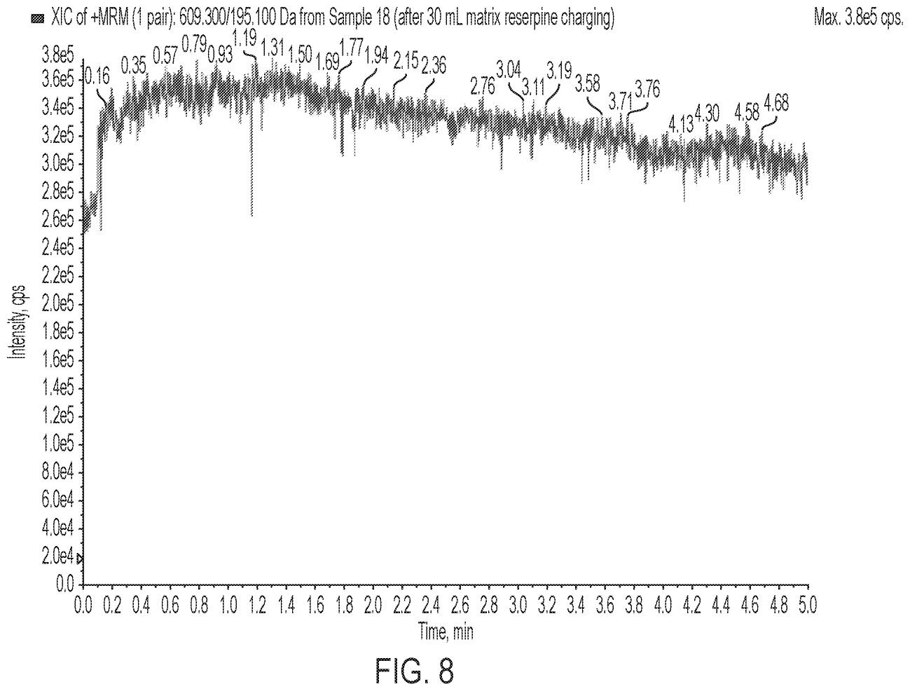

The experiments involved infusing a mixed extract of tea and arugula matrix in 10 mL increments, followed by baselining the performance of the mass spectrometer. Baselining experiments included charging tests conducted in MRM and Q1 mode to monitor for signal variation and changes to the Q1 peak width. FIG. 8 shows MRM charging data taken on the aforementioned triple quadrupole system after infusion of 30 mL of the sample matrix. The charging test involves operating the spectrometer in the negative ion mode for 5 minutes prior to switching to positive ion mode and tracking signal stability. This experiment was conducted without using a Q0 mass filter.

As shown in FIG. 8, substantial charging of Q1 was observed on this system after infusion of 30 mL of the food-based matrix, resulting in a signal that initially showed an increasing trend, followed by a decreasing trend. Over the course of a 5-minute experiment, the Q1 FWHM changed by more than 10% (not shown in this figure). It will be apparent to those having ordinary skill in the relevant arts that the example of FIG. 8 demonstrates severe charging effects that would impair mass analyzer performance.

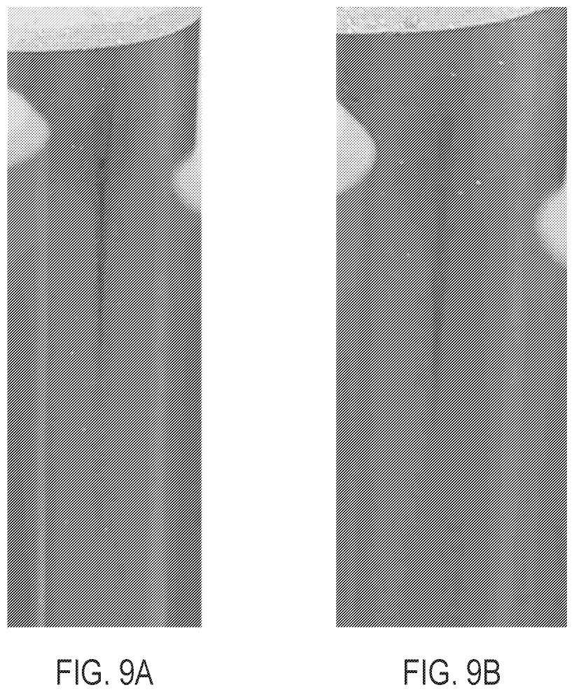

FIGS. 9A and 9B show digital photographs of debris patterns on one set of polls of the Q1 rod set after infusion of 30 mL of the food-based matrix. Substantial debris patterns were visible on one pair of Q1 rods, while the other pair were relatively clean (not shown).

### Example 2

A series of additional experiments were conducted using a Q0 mass filter configured with tee-bar electrodes, similar to the mass filter discussed in connection with FIGS. 3-4, while infusing the same food-based matrix into the mass spectrometer in order to establish mass windows associated with the contaminating ions.

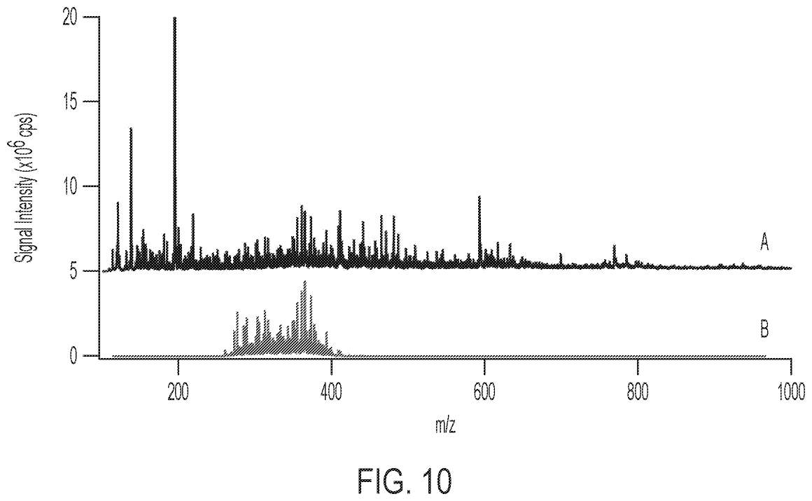

In a first experiment, the RF and the DC voltages applied to the tee bars and Q0 assembly were set to filter all charged species within the Q0 region, except for ions having an m/z ratio in the range of about 250 to about 400, which were then introduced into the Q1 mass analyzer as shown by the grey trace depicted in FIG. 10. This m/z window was selected after analyzing a list of 1033 typical pesticide MRM transitions. The average m/z value from the list was 312, with minimum and maximum values of 85 and 890.5, respectively. The m/z window of 250-400 would cover 583 of the 1033 (56%) MRM transitions typically monitored for pesticides on the 5500.

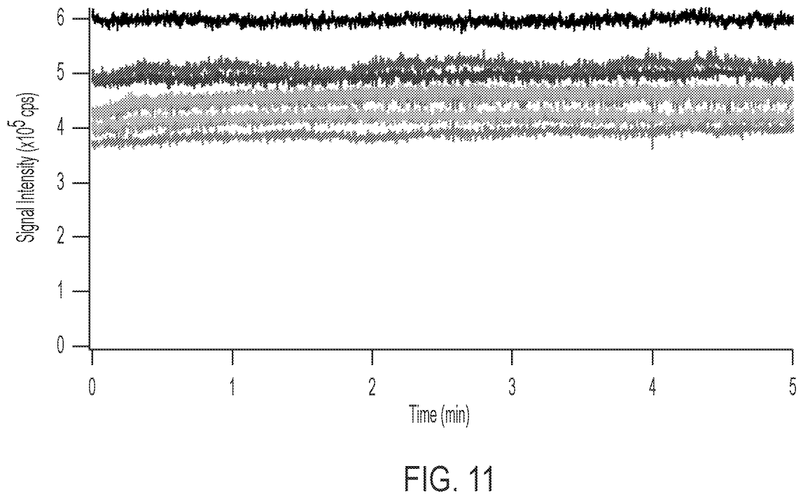

From the Q1 scans of FIG. 10, the measured total ion current dropped from approximately 4×10⁹ cps to 7×10⁸ cps when using the mass analyzer to scan a range of m/z of 100-1000, with and without an upstream mass filter establishing a bandpass window of m/z 250-400. When using the mass filter, it was possible to infuse 100 mL of matrix without significant charging of the mass analyzer. FIG. 11 shows signal intensity traces for a standard (reserpine ions) taken after each 10 mL matrix infusion. The total signal loss was less than 2×.

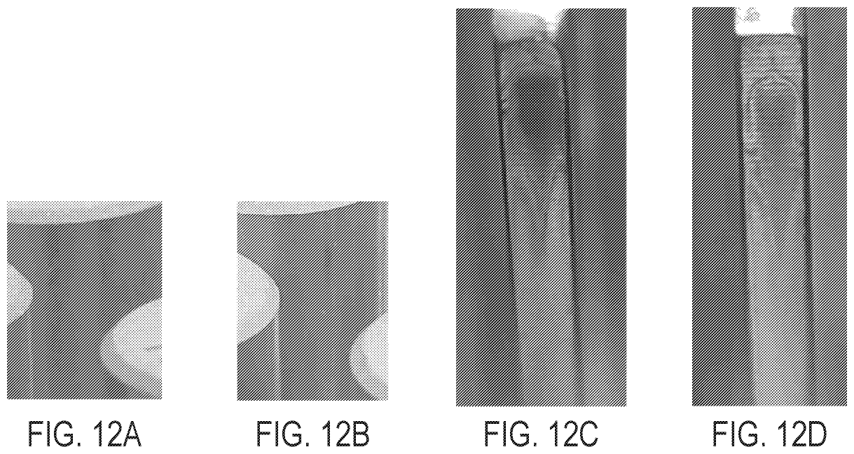

FIGS. 12A and 12B show digital photographs of the Q1 rod set indicating that no substantial build-up of debris was visible on the Q1 rods, despite infusion of 100 mL of the food-based matrix. FIGS. 12C and 12D show one pair of Q0 tee bars after infusion of 100 mL of the matrix with the bulk of debris deposited onto the surface in the entrance region. These results suggest that the bulk of debris that causes Q1 charging comes from charged species with m/z ratios that are outside of the range of m/z 250-400.

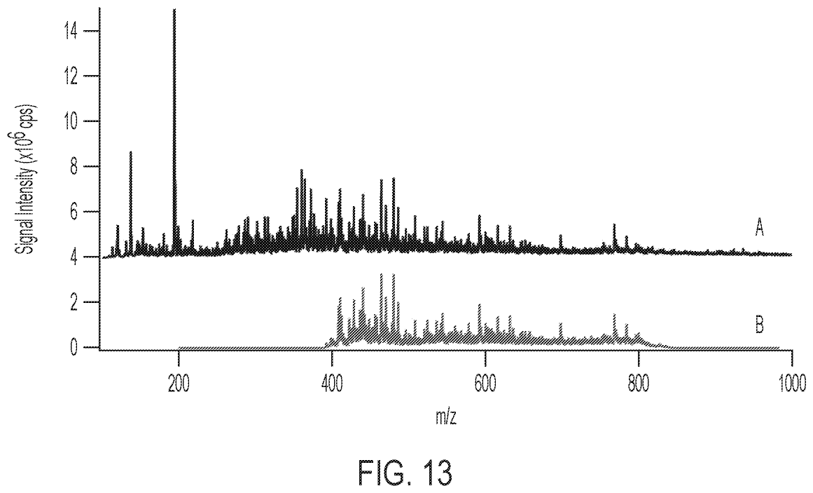

In a second set of experiments, the bandpass of the Q0 mass filter was set to a new m/z window spanning the range of 400 to 800 to obtain a mass spectrum of any ions having m/z ratios within this range, as shown in the grey trace depicted in FIG. 13. The TIC dropped from approximately 4×10⁹ cps to 1.7×10⁹ cps as a result of the tee bar windowing (not visible in this figure).

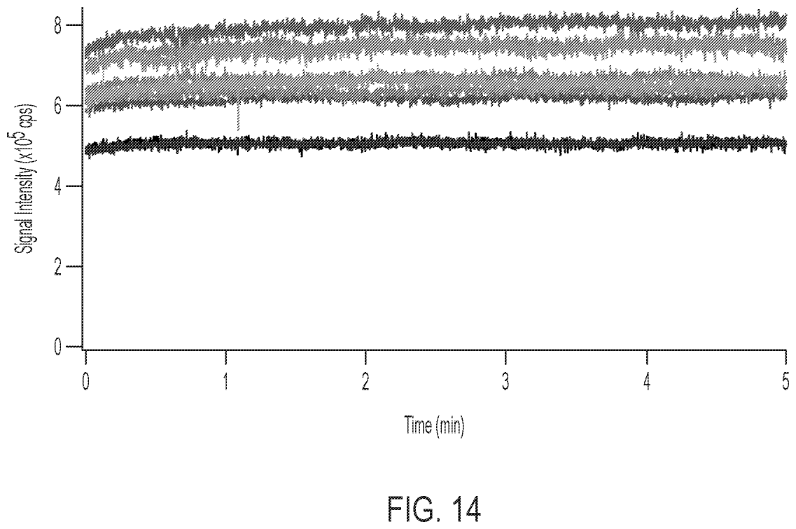

With the tee bars restricting the m/z range to 400-800, it was possible to spray 100 mL of food-based matrix without significant charging issues. Once again, FIG. 14 shows an overlay of intensity data taken for a reserpine standard after each 10 mL infusion of matrix. The total signal reduction was less than 2×.

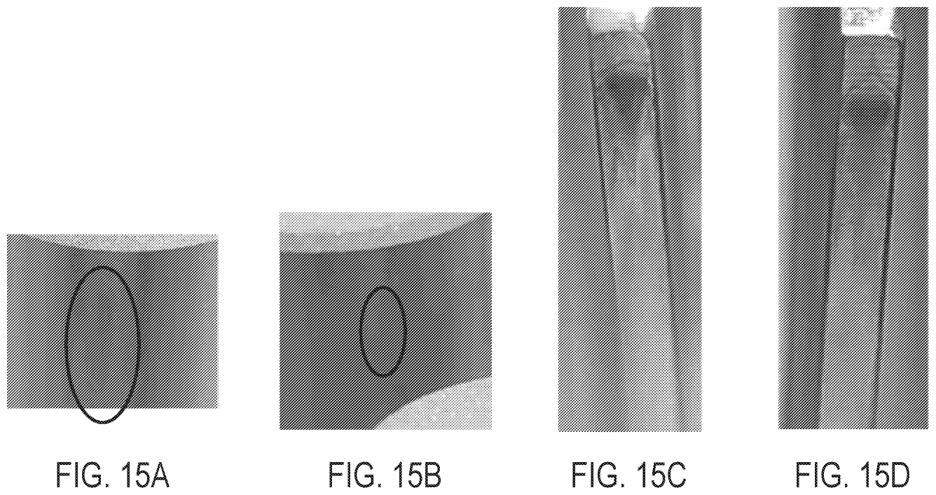

FIGS. 15A and 15B show photographs of the Q1 rods and FIGS. 15C and 15D show photographs of Q0 tee bars, illustrating that the bulk of the debris was deposited onto the Q0 tee bars, greatly reducing the amount of debris on the Q1 rods compared to the control experiment without tee bars filtering. A small deposit was visible on one pair of the Q1 rods, but the amount of the deposit was much less than what was observed after only 30 mL of matrix when the tee bars were not filtering. The gain in robustness was more than 3×, despite less than a 3× reduction in the total number of ions reaching the mass analyzer. These results suggest that it is not simply the TIC that matters with regard to contamination effects; certain types of debris are likely more detrimental than others.

The results thus far have shown that food-based matrices contain charged contaminating debris with m/z ratios that are outside the typical m/z range of 250-800. In order to confirm the mass range for these debris, an additional experiment was conducted in which the DC potential applied to the tee bars was turned off and the Q0 RF potential was increased to provide a low m/z cut-off at around m/z of 720. Under these conditions, significant charging was observed on the system after infusion of 40 mL of the food-based matrix. The Q1 peak width changed by more than 10% over the course of a 5 minute charging experiment.

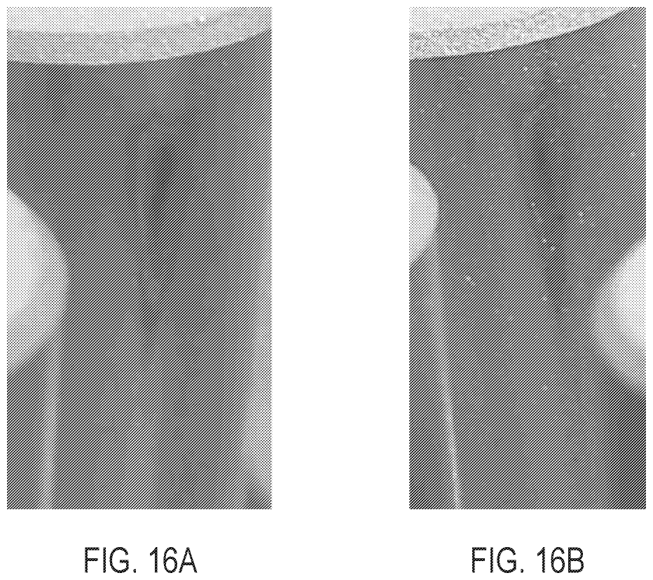

FIGS. 16A and 16B show digital photographs of one pair of the Q1 rods, demonstrating the presence of large deposits, similar to the original baseline data taken without using the tee bars (See, FIGS. 9A and 9B).

The above data shows that 1) the charged debris that is most responsible for causing Q1 charging when analyzing food-based matrices, such as extracts of tea and arugula, has m/z ratios greater than about 720 and 2) when the high m/z charged species are filtered prior to the Q1 analyzer, the instrument robustness is improved significantly, e.g., by more than a factor of 3 in the above experiments.

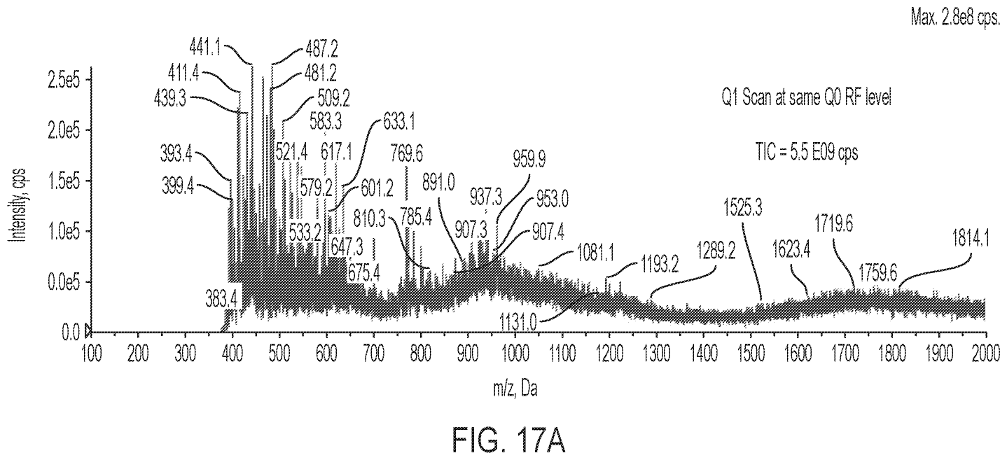

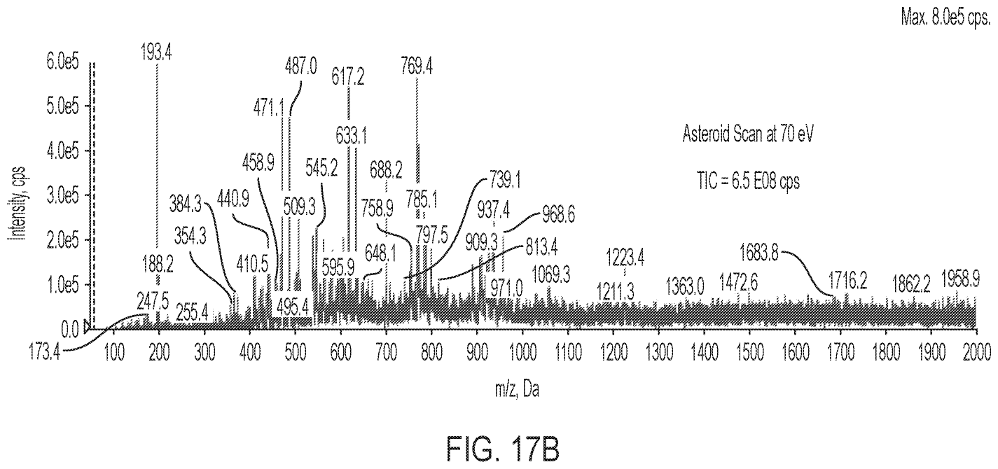

Additional experiments were conducted using the tea/arugula matrix to characterize the extent of large charged species with very high m/z. In a first set of experiments, the Q0 RF potential was set to 1560 V and the tee bars were turned off, as shown in FIG. 17A, where it is apparent that the Q0 RF level has resulted in a low m/z cut-off.

An elevated background was visible in the Q1 scan, but there was no indication of intense peaks in the region in which the m/z ratios are greater than approximately 1000. The mass analyzer on this triple quadrupole instrument was limited to m/z 2000, so a custom scan was used to try to characterize the magnitude of ion current with m/z ratios greater than 2000.

The Q1 mass analyzer was set to open and the instrument was operated in MS/MS mode with the precursor mass set to 2000. This resulted in a low m/z cut-off in the Q1 around m/z of 1500. The collision energy was then optimized to maximize the signal measured in Q3, and this required approximately 70 eV of energy. Under these conditions, numerous peaks were observed in the Q3 region, corresponding to fragments from larger charged cluster species, as shown in FIG. 17B.

Many of the peaks liberated in the Asteroid (ions with large m/z ratios, e.g., outside the upper mass limit of the MS) scan correspond to endogenous species from the food-based matrix that were also observed in the Q1 scan (FIG. 17A).

In previous Asteroid (ions with large m/z ratios, e.g., outside the upper mass limit of the MS) scan work with clean matrices, it was estimated that around 4% of the ion current for reserpine was trapped in large clusters. For the food-based matrices tested here, it appears that the Asteroid content can be much higher; the TIC from the Asteroid scan was 12% of the TIC from the Q1 scan. This means that these food-based matrices can create a disproportionate ion current contained in very large m/z clusters or droplets, contributing to mass analyzer contamination.

The pesticides that would normally be monitored for food-based matrices on a triple quadrupole spectrometer, such as Sciex 5500 series, includes 1033 MRMs, with Q1 m/z range extending from 85 to 890.5. Of these compounds, only 18 of them have m/z ratios of about 720, and therefore the windowing experiment described in FIGS. 13-15 show that the bulk of the charged debris comes from an m/z window that is greater than the m/z range needed for 98.3% of the pesticides. These results show: 1) food-based matrices contaminate triple quadrupole instruments faster than other common sample matrices, and 2) the main cause of degradation in the spectrometer's performance is Q1 charging.

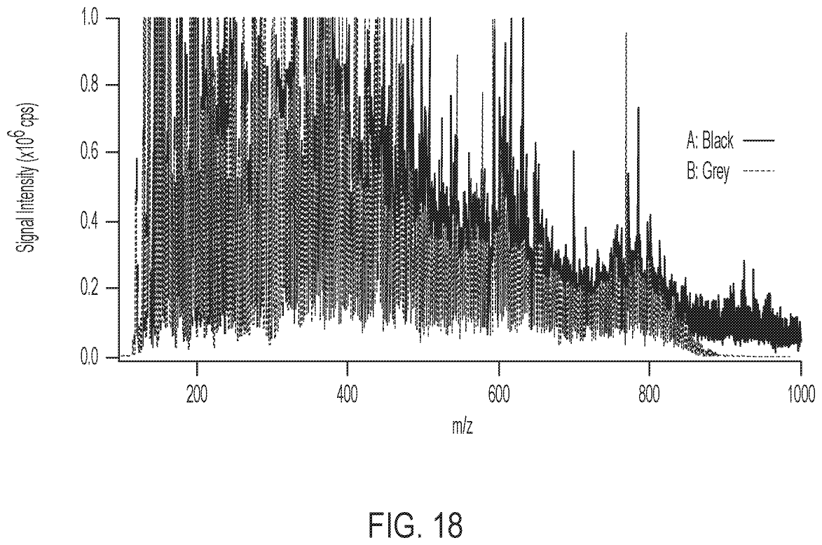

Furthermore, with a maximum pesticide m/z of 890.5, the examples described above suggest a new approach for pesticide analysis by including a mass filter with m/z cutoff greater than the maximum m/z of interest (in this case m/z 890.5). FIG. 18 shows an additional example where Q0 tee bars were used as a mass filter to eliminate all ions with m/z greater than 900. It was possible to spray 100 mL of food-based matrix with no substantial charging.

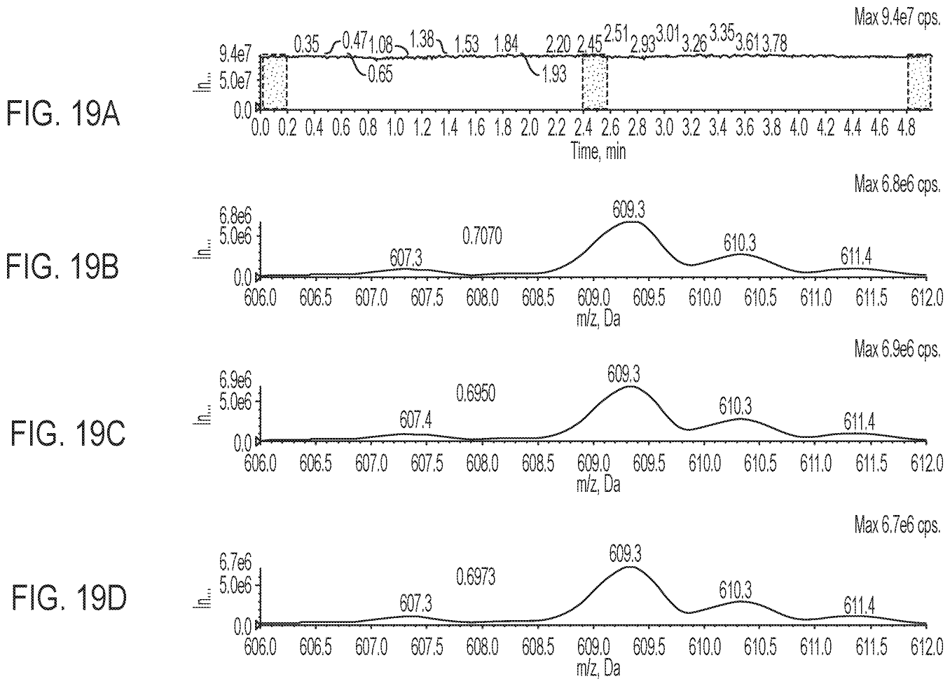

FIGS. 19A-19D show the TIC as well as the change in the Q1 peak width over the course of a 5-minute charging experiment after infusing 100 mL of matrix with the bandpass shown in FIG. 18, indicating that the reserpine signal and the Q1 peak shape remained constant after the infusion of 100 mL of the matrix.

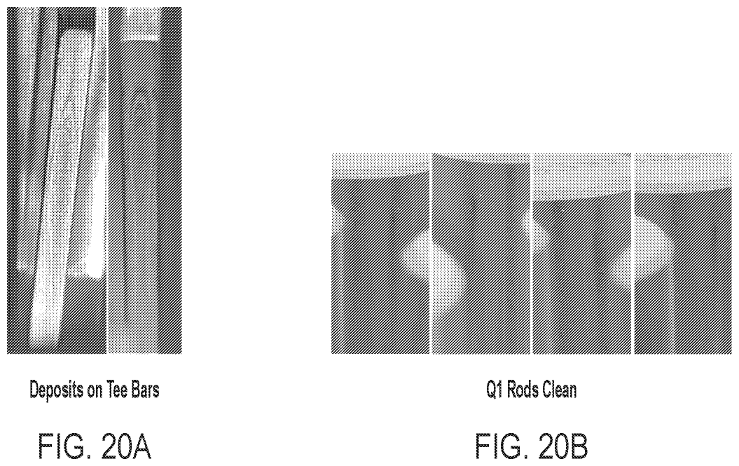

FIGS. 20A and 20B show digital photographs of the Q0 tee bars and Q1 rods, respectively. Again, the bulk of debris was deposited on the tee bars, and there was no significant debris accumulation on the Q1 mass analyzer.

### Example 3

In another series of experiments, a rat liver homogenate matrix was infused in 10 mL increments into a triple quadrupole mass spectrometer, followed by baselining the performance of the mass spectrometer, which involved charging tests conducted in MRM and Q1 mode to monitor for signal variation and changes to the Q1 peak width.

More specifically, the rat liver homogenate was prepared by mixing 1 part rat liver tissue with 10 parts PBS (phosphate buffer solution). The homogenate was precipitated according to one part tissue homogenate with 3 parts methanol (3.5 mL of homogenate and 10.5 mL of methanol), and the resultant precipitate was vortexed and centrifuged. The supernatant was removed and dried. It was then reconstituted with 15 mL of 80:20 Mobile Phase A:B (vortexed and sonicated to reconstitute), 0.1% formic acid, plus 5 mM ammonium formate. The reconstituted precipitate was filtered with 0.45 micron and 0.2 micron cellulose. The final dilution factor was 4.2 fold.

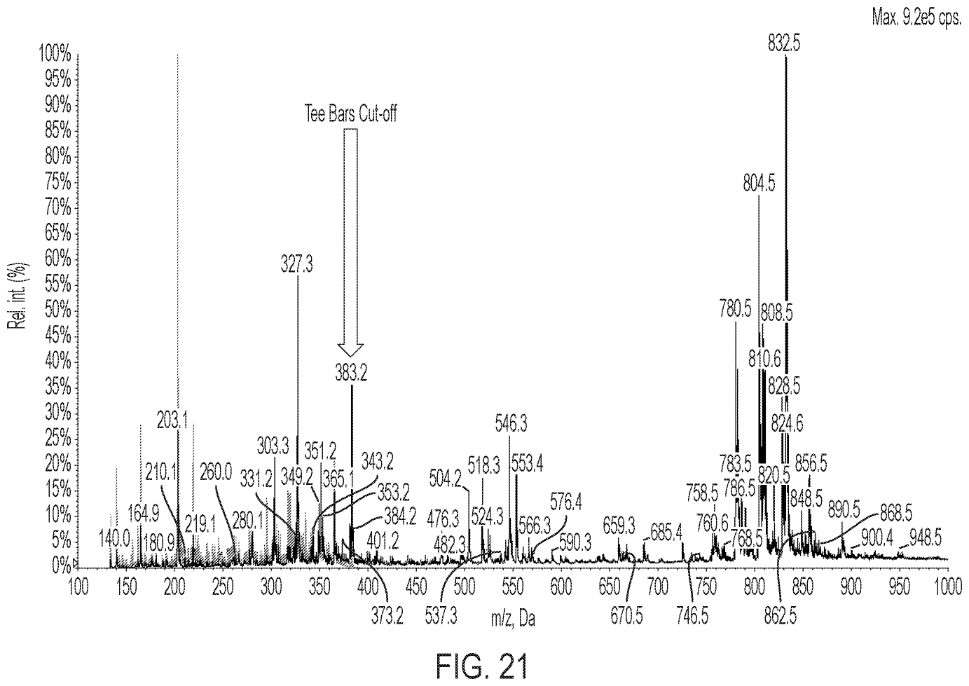

FIG. 21 shows the Q1 scans acquired when infusing the rat liver homogenate matrix. The mass spectrum marked as Trace 1 presents the mass spectrum that was obtained without using a Q0 mass filter. The mass spectrum marked as Trace 2 was obtained with the RF and the DC voltages applied to the tee bars and Q0 assembly set to filter all charged species within the Q0 region, except for ions having an m/z ratio less than 400, which were then introduced into the Q1 mass analyzer. This m/z window was selected based on user input that all analytes of interest have a m/z ratio of 314 or lower.

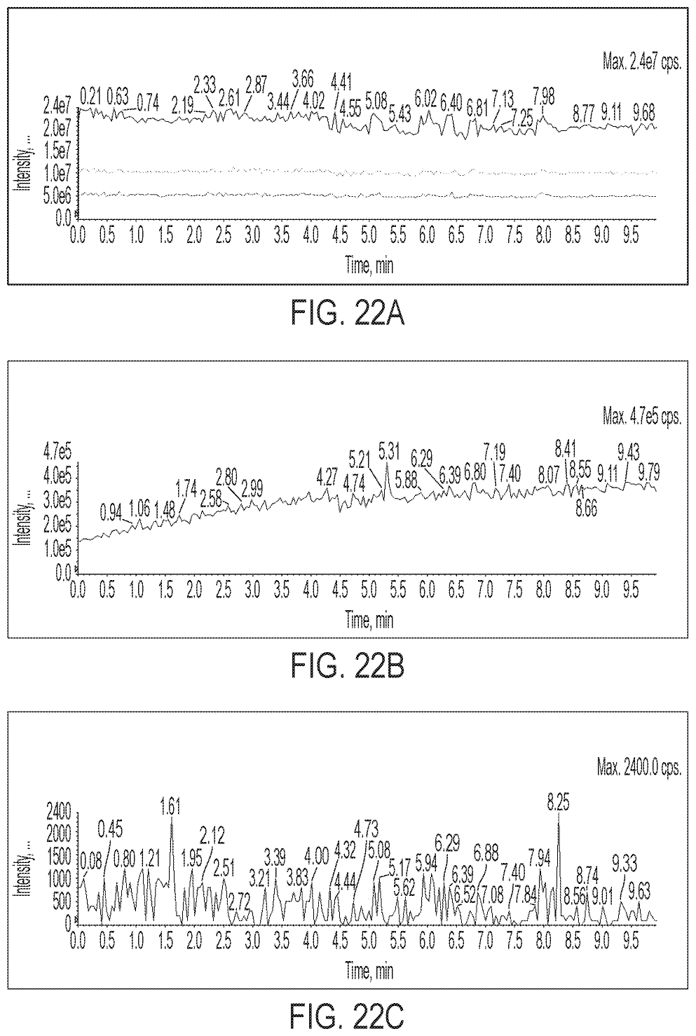

FIGS. 22A-C show Q1 charging data taken on Sciex 6500 triple quadrupole system after infusion of 40 mL of the sample matrix using no Q0 mass filter. This charging test involved operating the spectrometer in the negative ion mode for 10 minutes prior to switching to positive ion mode and tracking signal stability. FIG. 22A shows the data for m/z ratios 500 and greater, illustrating that minimal charging of Q1 was observed.

FIG. 22B shows the data for m/z 175 where the signal starts to increase over the course of the 10 minute run showing severe charging. FIG. 22C shows the data for m/z 59 illustrating a complete signal loss, which can be attributed to Q1 charging. These results show that after infusing 40 mL of the matrix with no tee bars, the system experienced severe charging for m/z ratios less than 300.

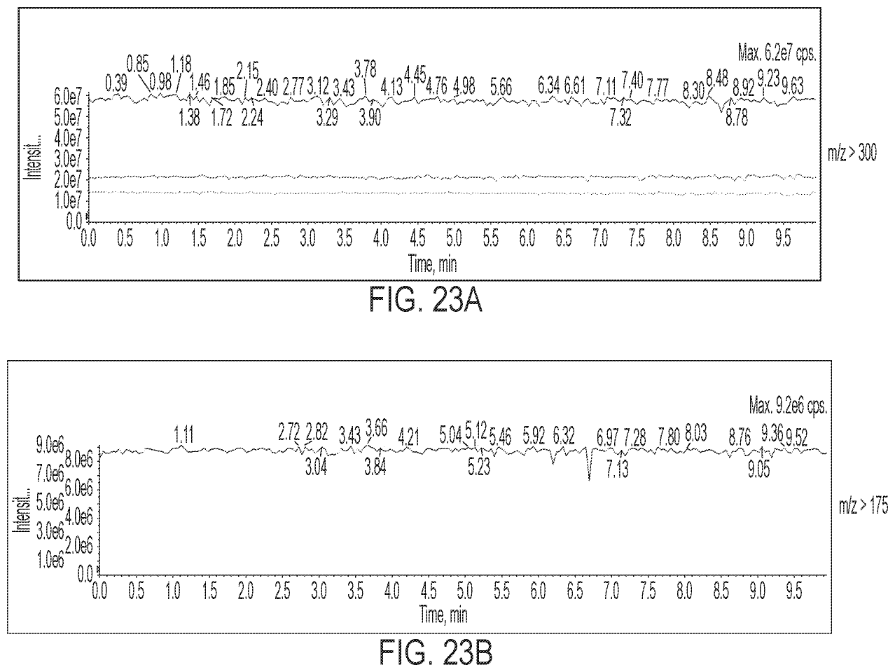

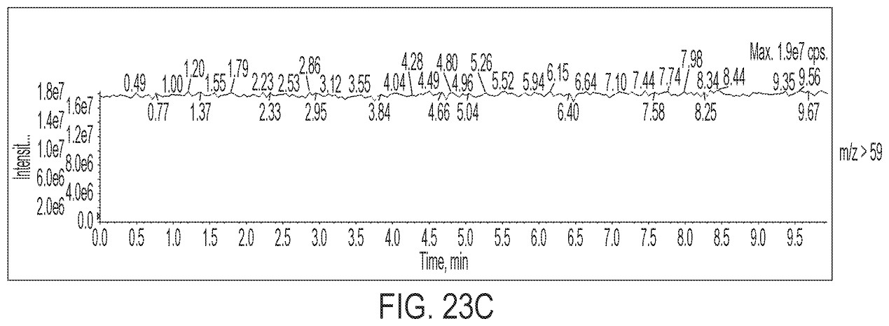

FIGS. 23A-C show Q1 charging data that was obtained using aforementioned triple quadrupole system after infusion of 40 mL of the sample matrix with the Q0 mass filter filtering above m/z 400. More specifically, FIG. 23A shows the charging data for m/z ratios 500 and greater, illustrating minimal charging. FIGS. 23B and 23C show, respectively, the charging data for m/z 175 and 59. These results show that after infusing 40 mL of the matrix with the tee bars filtering above m/z 400, there is minimal charging for any m/z ratio.

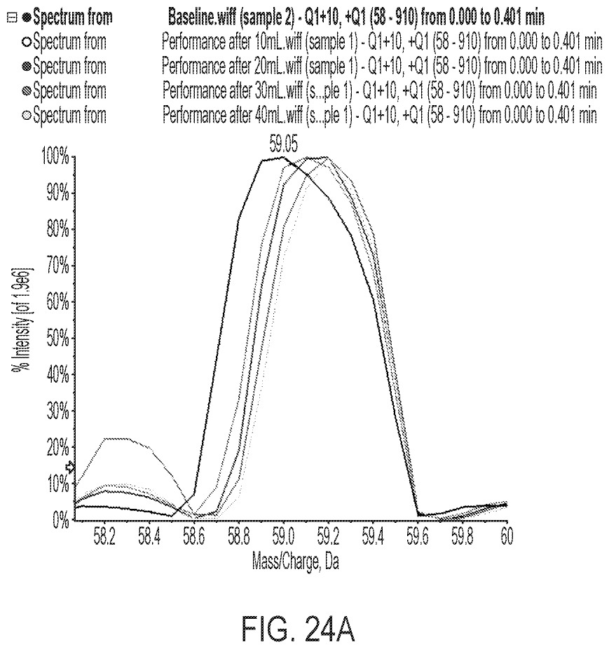

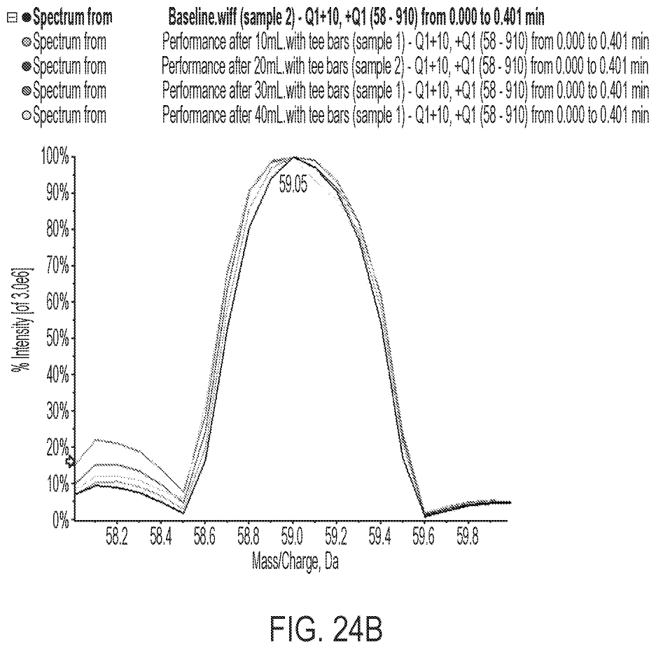

FIGS. 24A and 24B show overlays of the Q1 data for m/z of 59, taken after each 10 mL infusion of the matrix. The overlays shown in FIG. 24A correspond to data obtained when operating the mass spectrometer with no filtering by the tee bars. The peak width from the baseline (prior to starting the experiment) shown in the trace A begins to narrow after each infusion. The trace B shows the peak width after 40 mL infusion of the matrix, illustrating the Q1 FWHM changed by more than 26% from baseline. The overlays shown in FIG. 24B show the results when operating the tee bars to filter m/z ratios over 400. The peak widths show very minimal change after each 10 mL infusion, which tracks well with the charging data.

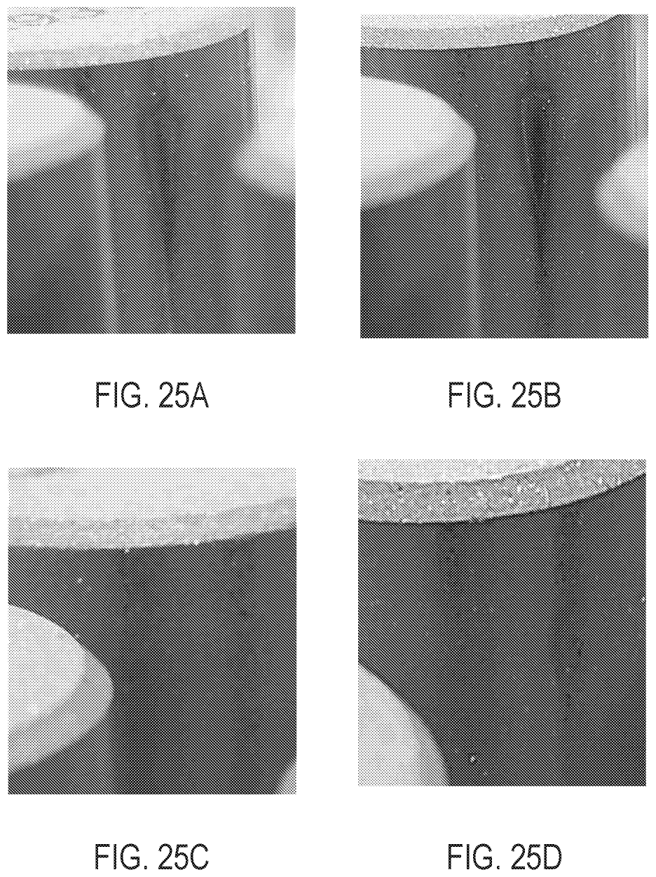

FIGS. 25A-D show digital photographs of debris patterns deposited on one set of poles of the Q1 rod set after infusion of 40 mL of the rat liver homogenate matrix. FIGS. 25A and 25B show that substantial debris patterns were visible on one pair of the Q1 rods where no tee bars were used. In contrast, as shown in FIGS. 25C and 25D, when the tee bars were set to filter above m/z 400, no visible debris deposits were present on the Q1 rods.

Those having ordinary skill in the art will appreciate that various changes can be made to the above embodiments without departing from the scope of the present teachings.

What is claimed is:

1. A mass spectrometer, comprising:
an atmospheric pressure ion source configured to receive a sample and ionize the sample to generate a plurality of ions,
a first mass filter positioned downstream of the ion source for receiving at least a portion of said plurality of ions,
a user interface for receiving information from a user regarding one or more m/z ratios or a range of m/z ratios of interest,
a controller in communication with said user interface and said first mass analyzer for receiving the information from said user interface regarding the m/z ratios or the range of m/z ratios of interest,
wherein said controller determines, based on the information received from the user interface, a maximum m/z ratio of interest for mass analysis and adjusts a bandpass window of said first mass analyzer such that said bandpass window has a high m/z cutoff greater than said maximum m/z ratio.

2. The mass spectrometer of claim 1, wherein said high m/z cutoff is separated from said maximum m/z ratio by a value in a range of about 10 to about 500.

3. The mass spectrometer of claim 1, wherein said first mass filter is disposed in a reduced pressure chamber.

4. The mass spectrometer of claim 4, wherein said reduced pressure chamber is maintained at a pressure in a range of about 2 mTorr to about 20 mTorr.

5. The mass spectrometer of claim 1, wherein said first mass filter comprises a plurality of rods arranged in a quadrupole configuration.

6. The mass spectrometer of claim 1, further comprising a second mass filter positioned downstream of said first mass filter for receiving ions transmitted through the first mass filter, said second mass filter having a bandpass window defining a range of m/z ratios that can be transmitted through said second mass filter.

7. The mass spectrometer of claim 6, wherein said controller is in communication with said second mass filter to adjust said bandpass window thereof to allow passage of one m/z ratios associated with ions received from said first mass filter.

8. The mass spectrometer of claim 7, wherein said controller is configured to shift the bandpass window of said second mass filter to allow passage of ions having a different m/z ratio received from said first mass filter.

9. A method of performing mass spectrometric analysis of a sample, comprising:
ionizing a sample to generate a plurality of ions,
introducing said plurality of ions into a mass filter positioned upstream of a mass analyzer, wherein said mass filter is configured to provide a high m/z cutoff greater than a maximum m/z ratio of ions associated with one or more analytes of interest in said sample so as to allow passage of said analyte ions while inhibiting passage of ions having m/z ratios above said high m/z cutoff,
performing a mass analysis of ions passing through said mass filter, and
wherein said high m/z cutoff is selected so as to reduce contamination of said downstream mass analyzer.

10. The method of claim 9, wherein said sample comprises a food-based sample;
optionally, wherein said food-based sample comprises tea;
optionally, wherein said food-based sample comprises arugula;
optionally, wherein processing of the food-based sample comprises utilizing an QUEChERS extraction method.

11. The method of claim 9, wherein said sample comprises a tissue sample;
optionally, wherein said tissue sample comprises a liver tissue homogenate.

12. The method of claim 9, wherein said step of performing mass analysis comprises introducing said ions passing through said mass filter into said downstream mass analyzer;
optionally, wherein said mass analyzer comprises a plurality of rods arranged in a multipole configuration.

13. The method of claim 9, wherein said high m/z cutoff is selected so as to reduce contamination of said multipole rods.

14. The method of claim 9, wherein the step of performing said mass analysis comprises causing fragmentation of at least a portion of the ions passing through said mass analyzer to generate a plurality of product ions; optionally, the method further comprising generating a mass spectrum of said product ions.

15. The method of claim 9, wherein said step of performing mass analysis comprises monitoring one or more MRM transitions of at least one of said analyte ions;
optionally, wherein said step of performing mass analysis comprises utilizing a quadrupole mass analyzer.

16. The method of claim 9, wherein said high m/z cutoff is about 700;
optionally, wherein said high m/z cutoff is about 1000.

17. The method of claim 9, wherein said mass filter is configured to provide a bandpass window for ion transmission.

18. The method of claim 17, wherein said bandpass window extends from about 20 amu to about 1250 amu.

19. The method of claim 9, further comprising processing said sample prior to said step of ionizing the sample.

20. The method of claim 9, wherein said one or more analytes of interest comprises at least one pesticide.
